(19) United States
(12) Patent Application Publication
(10) Pub. No.: US 2007/0155779 A1
(43) Pub. Date: Jul. 5, 2007

(54) BICYCLIC HETEROARYL COMPOUNDS AS PDE10 INHIBITORS

(75) Inventors: Patrick R, Verhoest, Old Lyme, CT (US); Christopher J. Helal, East Lyme, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/619,218

(22) Filed: Jan. 3, 2007

Related U.S. Application Data
(60) Provisional application No. 60/756,450, filed on Jan. 5, 2006.

(51) Int. Cl. A61K 31/4745; A61K 31/4439; C07D 471/02; C07D 403/14

(52) U.S. Cl. 514/303; 514/338; 546/118; 546/273.4

(57) ABSTRACT

The invention pertains to bicyclic heteroaryl compounds that serve as effective phosphodiesterase (PDE) inhibitors The invention also relates to compounds which are selective inhibitors of PDE-10. The invention further relates to intermediates for preparation of such compounds; pharmaceutical compositions comprising such compounds; and the use of such compounds in methods for treating certain central nervous system (CNS) or other disorders. The invention relates also to methods for treating neurodegenerative and psychiatric disorders, for example psychosis and disorders comprising deficient cognition as a symptom.

### CROSS REFERENCE TO RELATED APPLICATION

The present application claims benefit of U.S. Ser. No. 60/756/450 filed on Jan. 5, 2006, which is incorporated by reference herein in its entirety.

### FIELD OF THE INVENTION

The invention pertains to bicyclic heteroaryl compounds that serve as effective phosphodiesterase (PDE) inhibitors. The invention also relates to compounds which are selective inhibitors of PDE-10. The invention further relates to intermediates for preparation of such compounds; pharmaceutical compositions comprising such compounds; and the use of such compounds in methods for treating certain central nervous system (CNS) or other disorders, The invention relates also to methods for treating neurodegenerative and psychiatric disorders, for example psychosis and disorders comprising deficient cognition as a symptom.

### BACKGROUND OF INVENTION

Phosphodiesterases (PDEs) are a class of intracellular enzymes involved in the hydrolysis of the nucleotides cyclic adenosine monophosphate (cAMP) and cyclic guanosine monophosphates (cGMP) into their respective nucleotide monophosphates. The cyclic nucleotides cAMP and cGMP are synthesized by adenylyl and guanylyl cyclases, respectively, and serve as secondary messengers in several cellular pathways.

The cAMP and cGMP function as intracellular second messengers regulating a vast array of intracellular processes particularly in neurons of the central nervous system. In neurons, this includes the activation of cAMP and cGMP-dependent kinases and subsequent phosphorylation of proteins involved in acute regulation of synaptic transmission as well as in neuronal differentiation and survival. The complexity of cyclic nucleotide signaling is indicated by the molecular diversity of the enzymes involved in the synthesis and degradation of cAMP and cGMP. There are at least ten families of adenylyl cyclases, two of guanylyl cyclases, and eleven of phosphodiesterases. Furthermore, different types of neurons are known to express multiple isozymes of each of these classes, and there is good evidence for compartmentalization and specificity of function for different isozymes within a given neuron.

A principal mechanism for regulating cyclic nucleotide signaling is by phosphodiesterase-catalyzed cyclic nucleotide catabolism. There are 11 known families of PDEs encoded by 21 different genes. Each gene typically yields multiple splice variants that further contribute to the isozyme diversity. The PDE families are distinguished functionally based on cyclic nucleotide substrate specificity, mechanism(s) of regulation, and sensitivity to inhibitors. Furthermore, PDEs are differentially expressed throughout the organism, including in the central nervous system. As a result of these distinct enzymatic activities and localization, different PDEs' isozymes can serve distinct physiological functions. Furthermore, compounds that can selectively inhibit distinct PDE families or isozymes may offer particular therapeutic effects, fewer side effects, or both.

PDE10 is identified as a unique family based on primary amino acid sequence and distinct enzymatic activity. Homology screening of EST databases revealed mouse PDE10A as the first member of the PDE10 family of PDEs (Fujishige et at., J. Biol. Chem. 274:18438-18445, 1999; Loughney, K. et al., Gene 234:109-117, 1999). The murine homologue has also been cloned (Soderling, S. et al., Proc. Natl. Acad. Sci. USA 96:7071-7076, 1999)and N-terminal splice variants of both the rat and human genes have been identified (Kotera, J. et al., Biochem. Biophys. Res. Comm. 261-551-557, 1999; Fujishige, K. et al., Eur. J. Biochem. 266:1118-1127, 1999). There is a high degree of homology across species. The mouse PDE10A1 is a 779 amino acid protein that hydrolyzes both cAMP and cGMP to AMP and GMP, respectively. The affinity of PDE10 for cAMP (Km=0.05 μM) is higher than for cGMP (Km=3 μM). However, the approximately 5-fold greater Vmax for cGMP over cAMP has lead to the suggestion that PDE10 is a unique cAMP-inhibited cGMPase (Fujishige et al., J. Biol. Chem. 274:18438-18445, 1999).

The PDE10 family of polypeptides shows a lower degree of sequence homology as compared to previously identified PDE families and has been shown to be insensitive to certain inhibitors that are known to be specific for other PDE families. U.S. Pat. No. 6,350,603, incorporated herein by reference.

PDE10 also is uniquely localized in mammals relative to other PDE families. mRNA for PDE10 is highly expressed only in testis and brain (Fujishige, K. et al., Eur J Biochem. 266:1118-1127, 1999; Soderling, S. et al., Proc. Natl. Acad. Sci. 96:7071-7076, 1999; Loughney, K. et al., Gene 234:109-117, 1999). These initial studies indicated that within the brain PDE10 expression is highest in the striatum (caudate and putamen), n. accumbens, and olfactory tubercle. More recently, a detailed analysis has been made of the expression pattern in rodent brain of PDE10 mRNA (Seeger, T. F. et al., Abst. Soc. Neurosci. 26:345.10, 2000)and PDE10 protein (Menniti. F. S., Stick, C. A., Seeger, T. F., and Ryan, A. M. , immunohistochemical localization of PDE10 in the rat brain. William Harvey Research Conference ‘Phosphodiesterase in Health and Disease’. Porto, Portugal, Dec. 5-7, 2001).

A variety of therapeutic uses for PDE inhibitors has been reported including obtrusive lung disease, allergies, hypertension, angina, congestive heart failure, depression and erectile dysfunction (WO 01/41807 A2, incorporated herein by reference).

The use of selected benzimidazole and related heterocyclic compounds in the treatment of ischemic heart conditions has been disclosed based upon inhibition of PDE associated cGMP activity. U.S. Pat No. 5,693,652, incorporated herein by reference.

United States Patent Application Publication No. 2003/0032579 discloses a method for treating certain neurologic and psychiatric disorders with the selective PDE10 inhibitor papaverine. In particular, the method relates to psychotic disorders such as schizophrenia, delusional disorders and drug-induced psychosis; to anxiety disorders such as panic and obsessive-compulsive disorder; and to movement disorders including Parkinson's disease and Huntington's disease.

### SUMMARY OF THE INVENTION

The present invention provides for a compound of formula I or a pharmaceutical acceptable salt thereof, 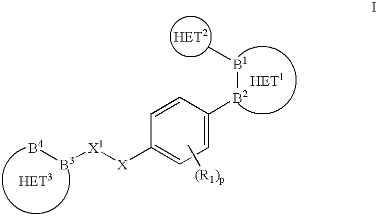
wherein HET¹ is selected from the group consisting of a monocyclic heteroaryl and a bicyclic heteroaryl, wherein said HET¹ may optionally be substituted with at least one R⁴;
HET² is a monocyclic heteroaryl, wherein said HET² may optionally be substituted with at least one R⁵;
HET³ is an 8 or 9 membered bicyclic heteroaryl, wherein said HET³ may optionally be substituted with at least one R⁶;
R¹ is selected from the group consisting of halogen, hydroxyl, cyano, C₁ to C₈ alkyl, C₂ to C₈ alkenyl, C₂ to C₈ alkynyl, C₁ to C₈ alkoxy, C₁ to C₈ haloalkyl, C₃ to C₈ cycloalkyl, C₂ to C₇ heterocycloalkyl, C₁ to C₈ alkylthio, —NR³R³, —O—CF₃, S(O)—R—; —C(O)—NR³R³, and C₁ to C₈ alkyl substituted with a heteroatom wherein the heteroatom is selected from the group consisting of nitrogen, oxygen and sulfur and wherein the heteroatom may be further substituted with one or more substituents selected from the group consisting of hydrogen, C₁ to C₈ alkyl, C₃ to C₈ cycloalkyl, C₂ to C₈ alkenyl, C₂ to C₈ alkynyl, and C₁ to C₈ haloalkyl;
each R² is independently selected from the group consisting of hydrogen, C₁ to C₈ alkyl, —C₃ to C₈ cycloalkyl-C₁ to C₈ alkyl, C₂ to C₈ alkenyl, C₂ to C₈ alkynyl, C₂ to C₈ alkenyl, C₁ to C₈ haloalkyl and C₃ to C₈ cycloalkyl;
each R³ is independently selected from the group consisting of hydrogen, C₁ to C₈ alkyl, C₂ to C₈ alkenyl, C₂ to C₈ alkynyl, C₁ to C₈ haloalkyl, C₃ to C₈ cycloalkyl;
each R⁴ is independently selected from the group consisting of halogen, hydroxyl, cyano, C₁ to C₈ alkyl, C₂ to C₈ alkenyl, C₂ to C₈ alkynyl, C₁ to C₈ alkoxy, C₃ to C₈ cycloalkyl, C₁ to C₈ alkylthio, C₁ to C₈ haloalkyl and C₁ to C₈ alkyl substituted with one or more substituents selected from the group consisting of —OR⁸, —NR⁸R⁸, and —SR⁸;
R⁵ is independently selected from the group consisting of halogen, hydroxyl cyano, —NR⁸R⁸, C₁ to C₈ alkyl, C₂ to C₈ alkenyl, C₁ to C₈ alkynyl, C₁ to C₈ alkoxy, C₃ to C₈ cycloalkyl, C₁ to C₈ alkylthio and C₁ to C₈ haloalkyl;
B¹ and B² are adjacent atoms in Het¹ which are independently selected from the group consisting of carbon and nitrogen;
B³ and B⁴ are adjacent atoms in Het³ wherein B² is carbon and B⁴ is nitrogen:
X and X¹ are each independently selected from the group consisting of oxygen, sulfur, —C(R₂)₂ and —NR₂, provided that at least one of X or X¹ is —C(R₂)₂;
wherein each R⁶ is independently selected from the group consisting of halogen, hydroxyl, cyano, C₁ to C₈ alkyl, C₂ to C₈ alkenyl, C₂ to C₈ alkynyl, C₁ to C₈ alkoxy, C₁ to C₈ cycloalkyl, C₁ to C₈ alkylthio, C₃ to C₈ haloalkyl, —NR⁷R⁷, —O—CF₃, —S(O)ₘ—R⁷, and —C(O)NR⁷R⁷, C₁ to C₈ alkyl substituted with a heteroatom wherein the heteroatom is selected from the group consisting of nitrogen, oxygen and sulfur and wherein the heteroatom may be further substituted with a substituent selected from the group consisting of hydrogen, C₁ to C₈ alkyl, C₁ to C₈ cycloalkyl. C₂ to C₈ alkenyl, C₂ to C₈ alkynyl and C₁ to C₈ haloalkyl;
or two R⁶s together with the atoms which they are attached may optionally form a C₄ to C₁₀ cycloalkyl, C₄ to C₁₀ cycloalkenyl, (4-10 membered) heterocycloalkyl or (4-10 membered) heterocycloalkenyl ring;
wherein each R⁷ is independently selected from the group consisting of hydrogen and C₁-C₈ alkyl;
wherein each R⁶ is independently selected from the group consisting of hydrogen, C₁ to C₈ alkyl, C₂ to C₈ alkenyl and C₂ to C₈ alkynyl;
n=0, 1 or 2; m=0, 1 or 2; and p=0, 1, 2, 3 or 4.

In one embodiment of the present invention HET³ is selected from the group consisting of: 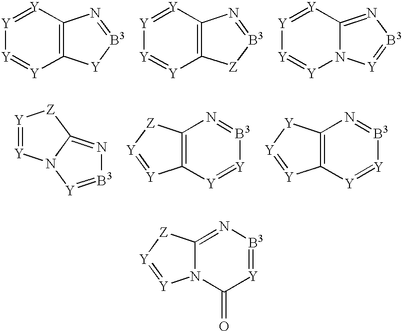
wherein each Y is independently selected from the group consisting of —CH, —CR⁶ or nitrogen, and Z is oxygen or sulfur.

In another embodiment of the present invention all Y's are independently —CH or —CR⁶.

In another embodiment of the present invention HET³ is selected from the group consisting of:

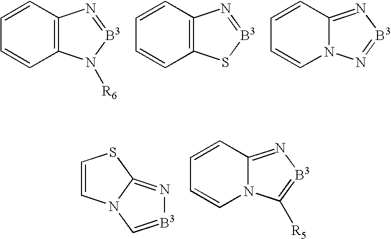

In another embodiment of the present invention HET¹ is a 5 membered heteroaryl.

In another embodiment of the present invention HET¹ is selected from the group consisting of pyrazolyl, isoxazolyl, triazolyl, oxazolyl, thiazolyl and imidazolyl.

In another embodiment of the present invention HET² is selected from the group consisting of 4-pyridyl, 4-pyridazinyl and isoxazolyl.

In another embodiment of the present invention HET² is 4-pyridyl.

In another embodiment of the present invention HET¹ is selected from the group consisting of:

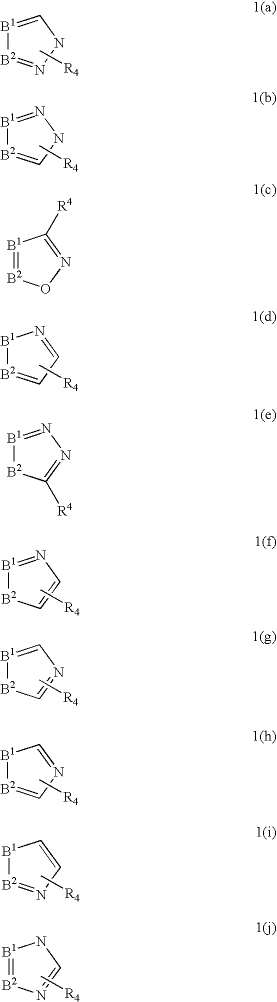

In another embodiment, in 1(a) above, B¹ and B² are carbon; in 1(b), B¹ and B² are carbon; in 1(c), B¹ and B² are carbon; in 1(d), B¹ is nitrogen and B² is carbon; in 1(e), B¹ is carbon and B² is nitrogen; in 1(f) B¹ is carbon and B² is nitrogen; in 1(g), B¹ is carbon and B² is nitrogen; in 1 (h), B¹ is nitrogen and B² is carbon; in 1 (i), B¹ is nitrogen and B² is carbon; and in 1(j), B¹ is carbon and B² is carbon.

In another embodiment of the present invention HET¹ is selected from the group 1(a) and HET² is 4-pyridyl

In another embodiment of the present invention X¹ is carbon and X is oxygen.

Compounds of the Formula I may have optical centers and therefore may occur in different enantiomeric and diastereomeric configurations. The present invention includes all enantiomers, diastereomers, and other stereoisomers of such compounds of the Formula I, as well as racemic compounds and racemic mixtures and other mixtures of stereoisomers thereof.

Pharmaceutically acceptable salts of the compounds of Formula I include the acid addition and base salts thereof.

Suitable acid addition salts are formed from acids which form non-toxic salts. Examples includes but are not limited to, the acetate, adipate, aspartate, benzoate, besylate, bicarbonate/carbonate, bisulphate/sulphate, borate, carnsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mandelates mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, salicylate, saccharate, stearate, succinate, sulfonate, stannate, tartrate, tosylate, trifluoroacetate and xinofoate salts.

Suitable base salts are formed from bases which form non-toxic salts. Examples include, but are not limited to, the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002).

Pharmaceutically acceptable salts of compounds of Formula I may be prepared by one or more of three methods:
(i) by reacting the compound of Formula I with the desired acid or base;
(ii) by removing an acid- or base-labile protecting group from a suitable precursor of the compound of Formula I or by ring-opening a suitable cyclic precursor, for example, a lactone or lactam, using the desired acid or base; or
(iii) by converting one salt of the compound of Formula I to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionization in the resulting salt may vary from completely ionised to almost non-ionised.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of the invention may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

Hereinafter all references to compounds of Formula I include references to salts. solvates, multi-component complexes and liquid crystals thereof and to solvates, multi-component complexes and liquid crystals of salts thereof.

The compounds of the invention include compounds of Formula I as hereinbefore defined, including all polymorphs and crystal habits thereof, prodrugs and isomers thereof (including optical, geometric and tautomeric isomers) as hereinafter defined and isotopically-labeled compounds of Formula I.

As indicated, so-called ‘prodrugs’ of the compounds of Formula I are also within the scope of the invention. Thus certain derivatives of compounds of Formula I which may have little or no pharmacological activity themselves can, when administered into or onto the body, be converted into compounds of Formula I having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in the compounds of Formula I with certain moieties known to those skilled in the art as ‘pro-moieties’ as described, for example, in Design of Prodrugs by H. Bundgaard (Elsevier, 1985).

Some examples of prodrugs in accordance with the invention include, but are not limited to,
(i) where the compound of Formula I contains a carboxylic acid functionality (—COOH), an ester thereof, for example, a compound wherein the hydrogen of the carboxylic acid functionality of the compound of Formula (I) is replaced by (C₁-C₈)alkyl;
(ii) where the compound of Formula I contains an alcohol functionality (—OH), an ether thereof, for example, a compound wherein the hydrogen of the alcohol functionality of the compound of Formula I is replaced by (C₁-C₆)alkanoyloxymethyl; and
(iii) where the compound of Formula I contains a primary or secondary amino functionality (—NH₂ or —NHR where R≠H), an amide thereof for example, a compound wherein, as the case may be, one or both hydrogens of the amino functionality of the compound of Formula I is/are replaced by (C₁-C₁₀) alkanoyl.

Further examples of replacement groups in accordance with the foregoing examples and examples of other prodrug types may be found in the aforementioned references.

Moreover, certain compounds of Formula I may themselves act as prodrugs of other compounds of Formula I.

Also included within the scope of the invention are metabolites of compounds of Formula I, that is, compounds formed in vivo upon administration of the drug. Some examples of metabolites in accordance with the invention include, but are not limited to,
(i) where the compound of Formula I contains a methyl group, an hydroxymethyl derivative thereof (—CH₃—>—CH₂OH):
(ii) where the compound of Formula I contains an alkoxy group, an hydroxy derivative thereof (—OR—>—OH);
(iii) where the compound of Formula I contains a tertiary amino group, a secondary amino derivative thereof (—NR¹R²—>—NHR¹ or —NHR²);
(iv) where the compound of Formula I contains a secondary amino group, a primary derivative thereof (—NHR¹—>NH₂)
(v) where the compound of Formula I contains a phenyl moiety, a phenol derivative thereof (—Ph—>—PhOH); and
(vi) where the compound of Formula I contains an amide group, a carboxylic acid derivative thereof (—CONH₂—>COOH).

Compounds of Formula I containing one or more asymmetric carbon atoms can exist as two or more stereoisomers. Where a compound of Formula I contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of Formula I containing, for example, an imino, keto, or oxime group, or so-called valence tautomerism in compounds that contain an aromatic moiety. It follows that a single compound may exhibit more than one type of isomerism.

Included within the scope of the present invention are all stereoisomers, geometric isomers and tautomeric forms of the compounds of Formula I, including compounds exhibiting more than one type of isomerism, and mixtures of one or more thereof. Also included are acid addition or base salts wherein the counterion is optically active, for example, d-lactate or l-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of Formula I contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art see, for example. Stereochemistry of Organic Compounds by E. L. Eliel and S. H. Wilen (Wiley, 1994).

The present invention includes all pharmaceutically acceptable isotopically-labelled compounds of Formula I wherein one or more atoms are replaced by atoms having the same atomic number, but an atomic mass or mass number different from the atomic mass or mass number which predominates in nature.

Examples of isotopes suitable for inclusion in the compounds of the invention include, but are not limited to, isotopes of hydrogen, such as ²H and ³H, carbon, such as ¹¹C, ¹³C and ¹⁴C, chlorine, such as ³⁶Cl, fluorine, such as ¹⁶F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of Formula I, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium. i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of Formula I can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g, D₂O, d₆-acetone, d₅-DMSO.

Specific embodiments of the present invention include the compounds exemplified in the Examples below and their pharmaceutically acceptable salts, complexes, solvates, polymorphs, steroisomers, metabolites, prodrugs, and other derivatives thereof,

This invention also pertains to a pharmaceutical composition for treatment of certain psychotic disorders and conditions such as schizophrenia, delusional disorders and drug induced psychosis; to anxiety disorders such as panic and obsessive-compulsive disorder; and to movement disorders including Parkinson's disease and Huntington's disease, comprising an amount of a compound of formula I effective in inhibiting PDE 10.

In another embodiment, this invention relates to a pharmaceutical composition for treating psychotic disorders and condition such as schizophrenia, delusional disorders and drug induced psychosis; anxiety disorders such as panic and obsessive-compulsive disorder; and movement disorders including Parkinson's disease and Huntington's disease, comprising an amount of a compound of formula I effective in treating said disorder or condition.

Examples of psychotic disorders that can be treated according to the present invention include, but are not limited to, schizophrenia, for example of the paranoid, disorganized, catatonic, undifferentiated, or residual type: schizophreniform disorder; schizoaffective disorder, for example of the delusional type or the depressive type; delusional disorder; substance-induced psychotic disorder, for example psychosis induced by alcohol, amphetamine, cannabis, cocaine, hallucinogens, inhalants, opioids, or phencyclidine; personality disorder of the paranoid type; and personality disorder of the schizoid type.

Examples of movement disorders that can be treated according to the present invention include but are not limited to selected from Huntington's disease and dyskinesia associated with dopamine agonist therapy, Parkinson's disease, restless leg syndrome, and essential tremor.

Other disorders that can be treated according to the present invention are obsessive/compulsive disorders, Tourette's syndrome and other tic disorders.

In another embodiment, this invention relates to a method for treating an anxiety disorder or condition in a mammal which method comprises administering to said mammal an amount of a compound of formula I effective in inhibiting PDE 10.

This invention also provides a method for treating an anxiety disorder or condition in a mammal which method comprises administering to said mammal an amount of a compound of formula I effective in treating said disorder or condition.

Examples of anxiety disorders that can be treated according to the present invention include, but are not limited to, panic disorder; agoraphobia; a specific phobia; social phobia; obsessive-compulsive disorder; postraumatic stress disorder; acute stress disorder; and generalized anxiety disorder.

This invention further provides a method of treating a drug addiction, for example an alcohol, amphetamine, cocaine, or opiate addiction, in a mammal, including a human, which method comprises administering to said mammal an amount of a compound of formula I effective in treating drug addiction.

This invention also provides a method of treating a drug addiction for example an alcohol, amphetamine, cocaine, or opiate addiction, in a mammal, including a human, which method comprises administering to said mammal an amount of a compound of formula I effective in inhibiting PDE10.

A “drug addiction”, as used herein, means an abnormal desire for a drug and is generally characterized by motivational disturbances such a compulsion to take the desired drug and episodes of intense drug craving.

This invention further provides a method of treating a disorder comprising as a symptom a deficiency in attention and/or cognition in a mammal, including a human, which method comprises administering to said mammal an amount of a compound of formula I effective in treating said disorder.

This invention also provides a method of treating a disorder or condition comprising as a symptom a deficiency in attention and/or cognition in a mammal, including a human, which method comprises administering to said mammal an amount of a compound of formula I effective in inhibiting PDE10.

This invention also provides a method of treating a disorder or condition comprising as a symptom a deficiency in attention and/or cognition in a mammal, including a human, which method comprises administering to said mammal an amount of a compound of formula I effective in treating said disorder or condition.

The phrase “deficiency in attention and/or cognition” as used herein in “disorder comprising as a symptom a deficiency in attention and/or cognition” refers to a subnormal functioning in one or more cognitive aspects such as memory, intellect, or learning and logic ability, in a particular individual relative to other individuals within the same general age population. “Deficiency in attention and/or cognition” also refers to a reduction in any particular individual's functioning in one or more cognitive aspects, for example as occurs in age-related cognitive decline.

Examples of disorders that comprise as a symptom a deficiency in attention and/or cognition that can be treated according to the present invention are dementia, for example Alzheimer's disease, multi-infarct dementia, alcoholic dementia or other drug-related dementia, dementia associated with intracranial tumors or cerebral trauma, dementia associated with Huntington's disease or Parkinson's disease, or AIDS-related dementia; delirium; amnestic disorder, post-traumatic stress disorder; mental retardation; a learning disorder, for example reading disorder, mathematics disorder, or a disorder of written expression, attention-deficit/hyperactivity disorder, and age-related cognitive decline.

This invention also provides a method of treating a mood disorder or mood episode in a mammal, including a human, comprising administering to said mammal an amount of a compound of formula I effective in treating said disorder or episode.

This invention also provides a method of treating a mood disorder or mood episode in a mammal, including a human, comprising administering to said mammal an amount of a compound of formula I effective in inhibiting PDE10.

Examples of mood disorders and mood episodes that can be treated according to the present invention include, but are not limited to, major depressive episode of the mild, moderate or severe type, a manic or mixed mood episode: a hypomanic mood episode; a depressive episode with atypical features; a depressive episode with melancholic features; a depressive episode with catatonic features; a mood episode with postpartum onset, post-stroke depression; major depressive disorder; dysthymic disorder; minor depressive disorder; premenstrual dysphoric disorder: post-psychotic depressive disorder of schizophrenia; a major depressive disorder superimposed on a psychotic disorder such as delusional disorder or schizophrenia, a bipolar disorder, for example bipolar I disorder, bipolar II disorder, and cyclothymic disorder.

This invention further provides a method of treating a neurodegenerative disorder or condition in a mammal, including a human, which method comprises administering to said mammal an amount of a compound of formula I effective in treating said disorder or condition.

This invention further provides a method of treating a neurodegenerative disorder or condition in a mammal, including a human, which method comprises administering to said mammal an amount of a compound of formula I effective in inhibiting PDE10.

As used herein, and unless otherwise indicated, a “neurodegenerative disorder or condition” refers to a disorder or condition that is caused by the dysfunction and/or death of neurons in the central nervous system. The treatment of these disorders and conditions can be facilitated by administration of an agent which prevents the dysfunction or death of neurons at risk in these disorders or conditions and/or enhances the function of damaged or healthy neurons in such a way as to compensate for the loss of function caused by the dysfunction or death of at-risk neurons. The term “neurotrophic agent” as used herein refers to a substance or agent that has some or all of these properties.

Examples of neurodegenerative disorders and conditions that can be treated according to the present invention include, but are not limited to, Parkinson's disease; Huntington's disease: dementia, for example Alzheimer's disease, multi-infarct dementia, AIDS-related dementia, and Fronto temperal Dementia; neurodegeneration associated with cerebral trauma; neurodegeneration associated with stroke, neurodegeneration associated with cerebral infarct; hypoglycemia-induced neurodegeneration; neurodegeneration associated with epileptic seizure; neurodegeneration associated with neurotoxin poisoning; and multi-system atrophy.

In one embodiment of the present invention, the neurodegenerative disorder or condition comprises neurodegeneration of striatal medium spiny neurons in a mammal, including a human.

In a further embodiment of the present invention, the neurodegenerative disorder or condition is Huntington's disease.

This invention also provides a pharmaceutical composition for treating psychotic disorders, delusional disorders and drug induced psychosis; anxiety disorders, movement disorders, mood disorders, neurodegenerative disorders and drug addiction, comprising an amount of a compound of formula I effective in treating said disorder or condition.

This invention also provides a method of treating a disorder selected from psychotic disorders, delusional disorders and drug induced psychosis; anxiety disorders, movement disorders, mood disorders, and neurodegenerative disorders, which method comprises administering an amount of a compound of formula I effective in treating said disorder.

This invention also provides a method of treating disorders selected from the group consisting of: dementia, Alzheimer's disease, multi-infarct dementia, alcoholic dementia or other drug-related dementia, dementia associated with intracranial tumors or cerebral trauma, dementia associated with Huntington's disease or Parkinson's disease, or AIDS-related dementia; delirium; amnestic disorder; post-traumatic stress disorder: mental retardation, a learning disorder, for example reading disorder, mathematics disorder, or a disorder of written expression; attention-deficit/hyperactivity disorder; age-related cognitive decline, major depressive episode of the mild, moderate or severe type; a manic or mixed mood episode; a hypomanic mood episode; a depressive episode with atypical features; a depressive episode with melancholic features; a depressive episode with catatonic features; a mood episode with postpartum onset, post-stroke depression; major depressive disorder: dysthymic disorder; minor depressive disorder, premenstrual dysphoric disorder; post-psychotic depressive disorder of schizophrenia; a major depressive disorder superimposed on a psychotic disorder comprising a delusional disorder or schizophrenia; a bipolar disorder comprising bipolar I disorder, bipolar II disorder, cyclothymic disorder, Parkinson's disease; Huntington's disease; dementia, Alzheimer's disease, multi-infarct dementia, AIDS-related dementia, Fronto temperal Dementia; neurodegeneration associated with cerebral trauma; neurodegeneration associated with stroke; neurodegeneration associated with cerebral infarct; hypoglycemia-induced neurodegeneration; neurodegeneration associated with epileptic seizure; neurodegeneration associated with neurotoxin poisoning; multi-system atrophy, paranoid, disorganized, catatonic, undifferentiated or residual type; schizophreniform disorder; schizoaffective disorder of the delusional type or the depressive type; delusional disorder; substance-induced psychotic disorder, psychosis induced by alcohol, amphetamine, cannabis, cocaine, hallucinogens, inhalants, opioids, or phencyclidine; personality disorder of the paranoid type; and personality disorder of the schizoid type.

This invention also provides a method of treating psychotic disorders, delusional disorders and drug induced psychosis; anxiety disorders, movement disorders, mood disorders, neurodegenerative disorders and drug addiction which method comprises administering an amount of a compound of formula I effective in inhibiting PDE10.

The term “alkyl” as used herein, unless otherwise indicated, includes saturated monovalent hydrocarbon radicals having straight or branched moieties. Examples of alkyl groups include, but are not limited to, methyl, ethyl, propyl, isopropyl, and t-butyl,

The term “alkenyl”, as used herein, unless otherwise indicated, includes alkyl moieties having at least one carbon-carbon double bond wherein alkyl is as defined above. Examples of alkenyl include, but are not limited to, ethenyl and propenyl.

The term “alkynyl”, as used herein, unless otherwise indicated, includes alkyl moieties having at least one carbon-carbon triple bond wherein alkyl is as defined above. Examples of alkynyl groups include, but are not limited to, ethynyl and 2-propynyl.

The term “alkoxy”, as used herein, unless otherwise indicated, as employed herein alone or as part of another group refers to an alkyl, groups linked to an oxygen atom.

The term “alkylthio” as used herein, unless otherwise indicated, employed herein alone or as part of another group includes any of the above alkyl groups linked through a sulfur atom.

The term “halogen” or “halo” as used herein alone or as part of another group refers to chlorine, bromine, fluorine, and iodine.

The term “haloalkyl” as used herein, unless otherwise indicated, refers to at least one halo group, linked to an alkyl group. Examples of haloalkyl groups include trifluoromethyl, difluoromethyl and fluoromethyl groups.

The term “cycloalkyl”, as used herein, unless otherwise indicated, includes non-aromatic saturated cyclic alkyl moieties wherein alkyl is as defined above. Examples of cycloalkyl include, but are not limited to, cyclopropyl, cyclobutyl, cyclopentyl, cyclohexyl, and cycloheptyl.

The term “aryl”, as used herein, unless otherwise indicated, includes an organic radical derived from an aromatic hydrocarbon by removal of one hydrogen, such as phenyl, naphthyl, indenyl, and fluorenyl. “Aryl” encompasses fused ring groups wherein at least one ring is aromatic.

The terms “heterocyclic”, “heterocycloalkyl”, and like terms, as used herein, refer to non-aromatic cyclic groups containing one or more heteroatoms, preferably from one to four heteroatoms, each preferably selected from oxygen, sulfur and nitrogen. The heterocyclic groups of this invention can also include ring systems substituted with one or more oxo moieties. Examples of non-aromatic heterocyclic groups are azirdinyl, azetidinyl, pyrrolidinyl, piperidinyl, azepinyl, piperazinyl, 1,2,3,6-tetrahydropyridinyl, oxiranyl, oxetanyl, tetrahydrofuranyl, tetrahydrothienyl, tetrahydropyranyl, tetrahydrothiopyranyl, morpholino, thiomorpholino, thioxanyl, pyrrolinyl, indolinyl, 2H-pyranyl, 4H-pyranyl, dioxanyl, 1,3-dioxolanyl, pyrazolinyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, quinolizinyl, quinuclidinyl, 1,4-dioxaspiro[4.5]decyl, 1,4-dioxaspiro[4,4]nonyl, 1,4-dioxaspiro[4.3]octyl, and 1,4-dioxaspiro[4.2]heptyl.

The term “heteroaryl”, as used herein, refers to aromatic groups containing one or more heteroatoms (preferably oxygen, sulfur and nitrogen), preferably from one to four heteroatoms. A multicyclic group containing one or more heteroatoms wherein at least one ring of the group is aromatic is a “heteroaryl” group. The heteroaryl groups of this invention can also include ring systems substituted with one or more oxo moieties. Examples of heteroaryl groups are pyridinyl, pyridazinyl, imidazolyl, pyrimidinyl, pyrazo yi, triazolyl, pyrazinyl, quinolyl, isoquinotyl, tetrazolyl, furyl, thienyl, isoxazolyl, thiazolyl, oxazolyl, isothiazolyl, pyrrolyl, indolyl, benzimidazolyl, benzoftiranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl,

Unless otherwise indicated, the term “one or more” substituents, or “at least one” substituent as used herein, refers to from one to the maximum number of substituents possible based on the number of available bonding sites.

Unless otherwise indicated, all the foregoing groups derived from hydrocarbons may have up to about 1 to about 20 carbon atoms (e.g. C₁-C₂₀ alkyl, C₂-C₂₀ alkenyl, C₃-C₂₀ cycloalkyl, 3-20 membered heterocycloalkyl; C₅-C₂₀ aryl, 5-20 membered heteroaryl, etc.) or 1 to about 15 carbon atoms (e.g., C₁-C₁₅ alkyl, C₂-C₁₅ alkenyl, C₃-C₁₅ cycloalkyl, 3-15 membered heterocycloalkyl, C₅-C₁₅ aryl, 5-15 membered heteroaryl, etc.) , or 1 to about 12 carbon atoms, or 1 to about 8 carbon atoms, or 1 to about 6 carbon atoms.

“Neurotoxin poisoning” refers to poisoning caused by a neurotoxin, A neurotoxin is any chemical or substance that can cause neural death and thus neurological damage. An example of a neurotoxin is alcohol, which, when abused by a pregnant female, can result in alcohol poisoning and neurological damage known as Fetal Alcohol Syndrome in a newborn. Other examples of neurotoxins include, but are not limited to, kainic acid, domoic acid, and acromelic acid; certain pesticides, such as DDT; certain insecticides, such as organophosphates; volatile organic solvents such as hexacarbons (e.g. toluene); heavy metals (e.g. lead, mercury, arsenic, and phosphorous); aluminum; certain chemicals used as weapons, such as Agent Orange and Nerve Gas; and neurotoxic antineoplastic agents.

As used herein, the term “'selective PDE10 inhibitor” refers to a substance, for example an organic molecule, that effectively inhibits an enzyme from the PDE10 family to a greater extent than enzymes from the PDE 1-9 families or PDE11 family. In one embodiment, a selective PDE10 inhibitor is a substance for example an organic molecule, having a Kᵢ for inhibition of PDE10 that is less than or about one-tenth the Kᵢ that the substance has for inhibition of any other PDE enzyme. In other words, the substance inhibits PDE10 activity to the same degree at a concentration of about one-tenth or less than the concentration required for any other PDE enzyme.

In general, a substance is considered to effectively inhibit PDE10 activity if it has a Kᵢ of less than or about 10 μM, preferably less than or about 0.1 μM.

A “selective PDE10 inhibitor” can be identified, for example, by comparing the ability of a substance to inhibit PDE10 activity to its ability to inhibit PDE enzymes from the other PDE families. For example, a substance may be assayed for its ability to inhibit PDE10 activity, as well as PDE1A, PDE1B, PDE1C, PDE2, PDE3A, PDE3B, PDE4A, PDE4B, PDE4C, PDE4D, PDE5, PDE6, PDE7, PDE8, PDE9, and PDE11.

The term “treating”, as in “a method of treating a disorder”, refers to reversing, alleviating, or inhibiting the progress of the disorder to which such term applies, or one or more symptoms of the disorder. As used herein, the term also encompasses, depending on the condition of the patient, preventing the disorder, including preventing onset of the disorder or of any symptoms associated therewith, as well as reducing the severity of the disorder or any of its symptoms prior to onset. “Treating” as used herein refers also to preventing a recurrence of a disorder.

For example, “treating schizophrenia, or schizophreniform or schizoaffective disorder” as used herein also encompasses treating one or more symptoms (positive, negative, and other associated features) of said disorders, for example treating, delusions and/or hallucination associated therewith. Other examples of symptoms of schizophrenia and schizophreniform and schizoaffective disorders include disorganized speech, affective flattening, alogia, anhedonia, inappropriate affect, dysphoric mood (in the form of, for example, depression, anxiety or anger), and some indications of cognitive dysfunction.

The term “mammal”, as used herein, refers to any member of the class “Mammalia”, including, but not limited to, humans, dogs, and cats.

The compound of the invention may be administered either alone or in combination with pharmaceutically acceptable carriers, in either single or multiple doses. Suitable pharmaceutical carriers include inert solid diluents or fillers, sterile aqueous solutions and various organic solvents, The pharmaceutical compositions formed thereby can then be readily administered in a variety of dosage forms such as tablets, powders, lozenges, liquid preparations, syrups, injectable solutions and the like. These pharmaceutical compositions can optionally contain additional ingredients such as flavorings, binders, excipients and the like. Thus, the compound of the invention may be formulated for oral, buccal, intranasal, parenteral (e.g. intravenous, intramuscular or subcutaneous), transdermal (e.g. patch) or rectal administration, or in a form suitable for administration by inhalation or insufflation.

For oral administration, the pharmaceutical compositions may take the form of, for example, tablets or capsules prepared by conventional means with pharmaceutically acceptable excipients such as binding agents (e.g. pregelatinized maize starch, polyvinylpyrrolidone or hydroxypropyl methylcellilose), fillers (e.g. lactose, microcrystalline cellulose or calcium phosphate); lubricants (e.g. magnesium stearate, talc or silica); disintegrants (e.g. potato starch or sodium starch glycolate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, methyl cellulose or hydrogenated edible fats); emulsifying agents (e.g. lecithin or acacia); non-aqueous vehicles (e.g. almond oil, oily esters or ethyl alcohol); and preservatives (e.g. methyl or propyl p-hydroxybenzoates or sorbic acid).

For buccal administration, the composition may take the form of tablets or lozenges formulated in conventional manner.

The compounds of the invention may be formulated for parenteral administration by injection, including using conventional catheterization techniques or infusion. Formulations for injection may be presented in unit dosage form. e.g. in ampules or in multi-dose containers, with an added preservative. They may take such forms as suspensions, solutions or emulsions in oily or aqueous vehicles, and may contain, formulating agents such as suspending, stabilizing and/or dispersing agents. Alternatively, the active ingredient may be in powder form for reconstitution with a suitable vehicle, e.g. sterile pyrogen-free water, before use.

When a product solution is required, it can be made by dissolving the isolated inclusion complex in water (or other aqueous medium) in an amount sufficient to generate a solution of the required strength for oral or parenteral administration to patients. The compounds may be formulated for fast dispersing dosage forms (fddf), which are designed to release the active ingredient in the oral cavity. These have often been formulated using rapidly soluble gelatin-based matrices. These dosage forms are well known and can be used to deliver a wide range of drugs. Most fast dispersing dosage forms utilize gelatin as a carrier or structure-forming agent. Typically, gelatin is used to give sufficient strength to the dosage form to prevent breakage during removal from packaging, but once placed in the mouth, the gelatin allows immediate dissolution of the dosage form. Alternatively, various starches are used to the same effect.

The compounds of the invention may also be formulated in rectal compositions such as suppositories or retention enemas, e.g. containing conventional suppository bases such as cocoa butter or other glycerides.

For intranasal administration or administration by inhalation, the compound of the invention is conveniently delivered in the form of a solution or suspension from a pump spray container that is squeezed or pumped by the patient or as an aerosol spray presentation from a pressurized container or a nebulizer, with the use of a suitable propellant, e.g. dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made e.g. from gelatin) for use in an inhaler or insufflator may be formulated containing a powder mix of a compound of the invention and a suitable powder base such as lactose or starch.

Aerosol formulations for treatment of the conditions referred to above (e.g. migraine) in the average adult human are preferably arranged so that each metered dose or “puff” of aerosol contains about 20 mg to about 1000 mg of the compound of the invention. The overall daily dose with an aerosol will be within the range of about 100 mg to about 10 mg. Administration may be several times daily, e.g. 2, 3, 4 or 8 times, giving for example, 1, 2 or 3 doses each time.

A proposed daily dose of the compound of the invention for oral, parenteral, rectal or buccal administration to the average adult human for the treatment of the conditions referred to above is from about 0.01 mg to about 2000 mg, preferably from about 0.1 mg to about 200 mg of the active ingredient of formula I per unit dose which could be administered, for example, 1 to 4 times per day

Assay methods are available to screen a substance for inhibition of cyclic nuticleotide hydrolysis by the PDE10 and the PDEs from other gene families. The cyclic nucleotide substrate concentration used in the assay is ⅓ of the Kₘ concentration, allowing for comparisons of IC₅₀ values across the different enzymes. PDE activity is measured using a Scintillation Proximity Assay (SPA)-based method as previously described (Fawcett et al., 2000), The effect of PDE inhibitors is determined by assaying a fixed amount of enzyme (PDEs 1-11) in the presence of varying substance concentrations and low substrate, such that the IC₅₀ approximates the Kᵢ (cGMP or cAMP in a 3:1 ratio unlabelled to [³H]-labeled at a concentration of ⅓ Km).). The final assay volume is made up to 100 μl with assay buffer [20 mM Tris-HCl pH 7.4, 5 mM MgCl₂, 1 mg/ml bovine serum albumin]. Reactions are initiated with enzyme, incubated for 30-60 min at 30° C. to give <30% substrate turnover and terminated with 50 μl yttrium silicate SPA beads (Amersham) (containing 3 mM of the respective unlabelled cyclic nucleotide for PDEs 9 and 11). Plates are re-sealed and shaken for 20 min, after which the beads were allowed to settle for 30 minutes in the dark and then counted on a TopCount plate reader (Packard, Meriden, Conn.). Radioactivity units can be converted to percent activity of an uninhibited control (100%), plotted against inhibitor concentration and inhibitor IC₅₀ values can be obtained using the “Fit Curve’ Microsoft Excel extension.

Using such assay, compounds of the present invention were determined to have an IC₅₀ for inhibiting PDE10 activity of less than about 10 micromolar.

This invention also pertains to the preparation of compounds of formula I.

The schemes below depict various methods of preparing the compounds of the present invention. It should be noted that various substituents illustrated in the schemes (e.g, R₁, X, Y, etc.) are for illustrated purposes only and should not be confused with and may be independent of those recited above and in the claims.

Scheme 1 depicts the preparation of the pyrazole class of compounds of this invention. Alkylation of a substituted phenol with 2-Chloromethyl-1-methyl-1-H-benzoimidazole provides the desired ether. Other heteroaromatic benzyl chlorides and be substituted and prepared by those skilled in the art. Hydrolysis of the ester and treatment with thionyl chloride provides the desired acid chloride. Addition of O,N-dimethyl hydroxyl amine hydrochloride provides the Weinreb amide for coupling (Weinreb et al. Tet Lett, 1981, 22(39) 3815). Alternatively, the Weinreb amide can be formed by direct coupling to the acid with carbonyl diimidazole and the amide. Anion generation with 4-picoline and LDA followed by addition of the Weinreb amide affords the ketone. The ketone can then be treated with dimethoxymethyl-dimethyl amine at reflux to form the enaminone intermediate. Treatment with various hydrazines affords the pyrazole analogues. A variety of ratios of the two isomers were obtained. These isomers were separated via, crystallization, Biotage MPLC, preparative TLC or preparative HPLC. This reaction scheme is general for a variety of starting substituted phenols; substituted quinolines and substituted hydrazines. Substituted pyrazoles can also be formed by alkylation of the N—H pyrazole with an appropriate base (ie NaH, Cs₂CO₃) and an electophile (R-I etc.).

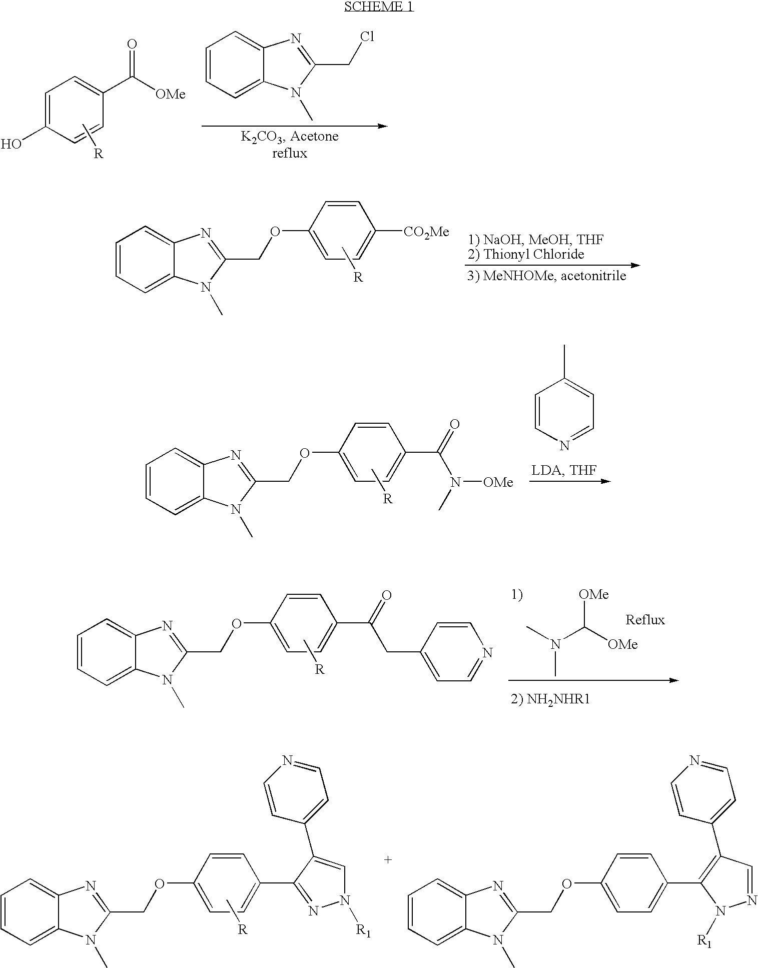

Alternatively, the substituted pyrazole compounds can be prepared by alkylation of the NH pyrazole. One set of conditions is the utilization of cesium carbonate as the base with an alkyl halide as the electrophile in a solvent such as dimethyl formamide. Some reactions require heating.

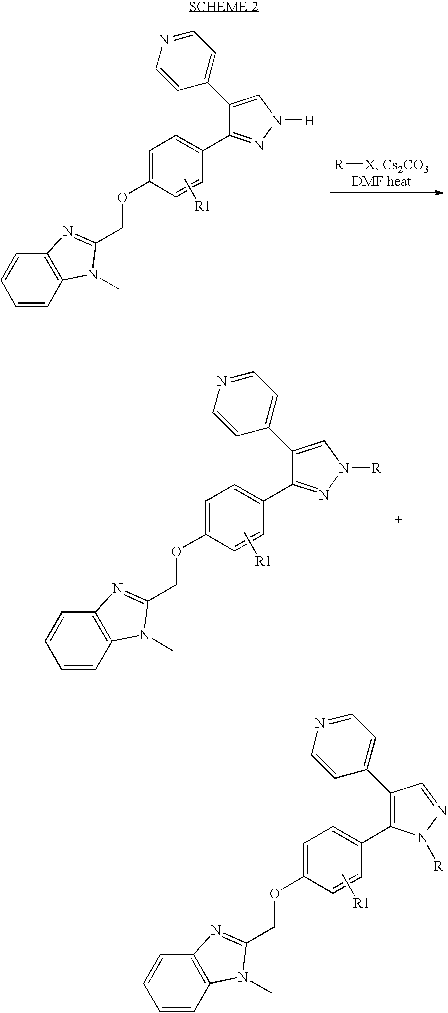

As depicted in Scheme 3 a variety of heterocycles can be prepared from the enaminone intermediate. Pyrimidines can be prepared by heating with substituted formamides in the presence of ethanol and sodium ethoxide. Isoxazoles are prepared by heating the enaminone with hydroxyl amine in methanol/acetic acid. Only one isomer in the isoxazole case is formed. By heating with amino pyroles, amino imidazoles or amino triazoles, 6-5 bicyclic systems can be formed.

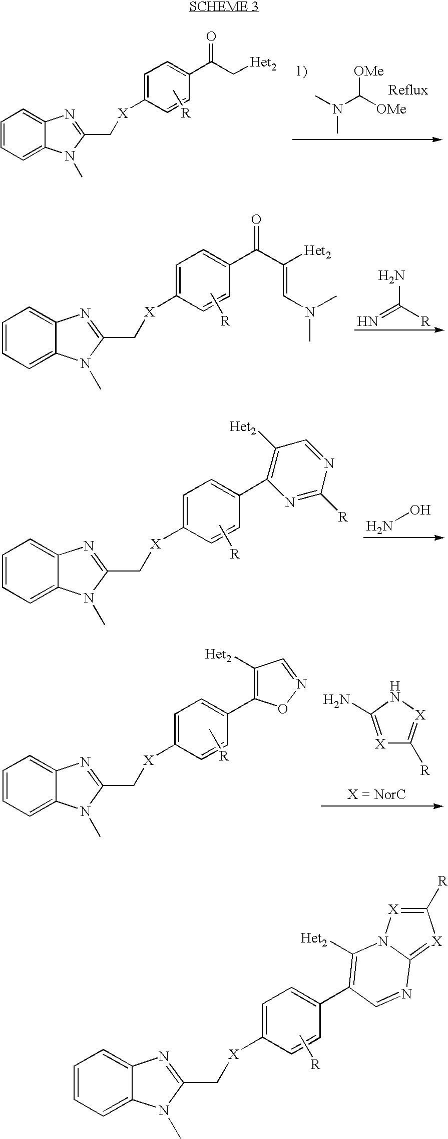

A variety of 4-pyridyl heterocyclic replacements can be prepared according to scheme 4. Methyl heterocycles such as 3,5-dimethyl isoxazole and methyl pyridazine can be deprotated with lithium diisopropyl amide and added to a Weinreb amide (Weinreb et al, Tet Lett., 1981 22(39) 3815) to provide the desired ketone. Sequential treatment with dimethoxymethyl-dimethyl amine and a hydrazine provides the heterocyclic pyrazoles. Pyrimidines and isoxazoles can also be prepared as described in Scheme 1, 2 and 3.

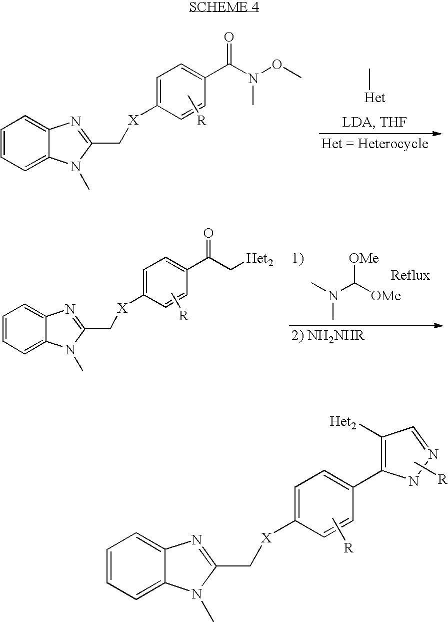

N-pyridyl pyrazoles can be prepared according to Scheme 5. The starting ketones are prepared by alkylation of the phenol as depicted in Scheme 1. Treatment of the ketone with dimethoxymethyl-dimethyl amine followed by addition of 4-pyridyl hydrazine (see J. Med. Chem. 2002, 45(24) 5397), provides the desired compounds. Other heterocyclic replacements for 4-pyridyl can be prepared by using the requisite hydrazine.

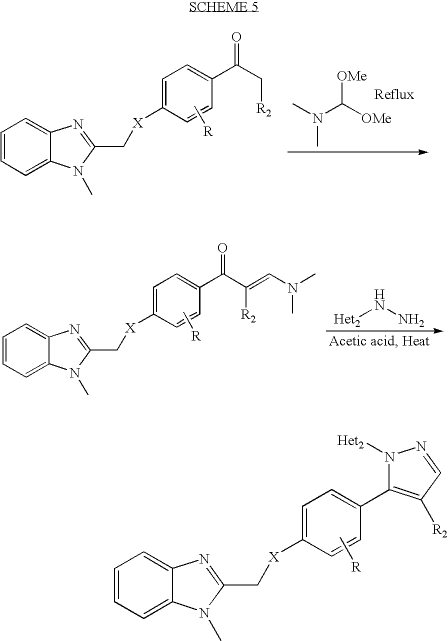

The benzyl intermediates can be prepared by the method shown in scheme 1. The benzyl ether can be removed via treatment with hydrogen gas over a palladium catalyst such as palladium on carbon or palladium hydroxide in a variety of solvents. The phenol can then be alkylated using an benylic chloride in acetone heating with potassium carbonate. Also Mitsunobu chemistry (Hughes, D. L., The Mitsunobu Reaction, Organic Reactions, Vol. 42. 1992, New York, 335-656.) can be applied to couple the phenol with alcohols.

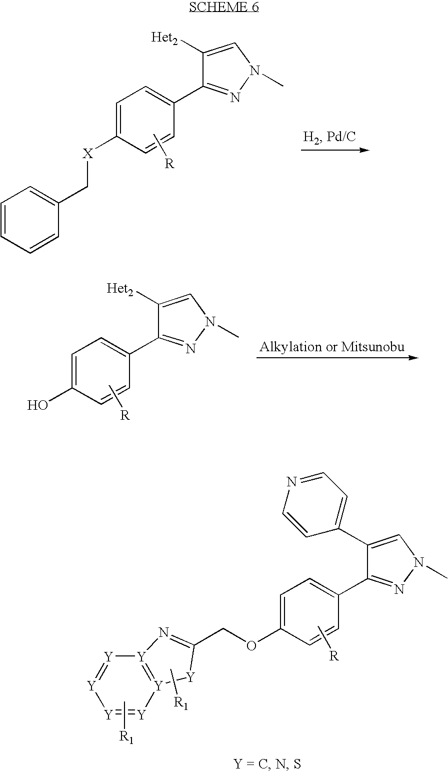

Many benzylic halides or alcohols are commercially available or are known in the literature. General ways to make these intermediates by those skilled in the art are reduction of an ester, acid or aldehyde to form an alcohol. One general procedure is the oxidation of a benylic site with selenium dioxide to provide an aldehyde that is subsequentially reduced with sodium borohydride. Benzylic halide can be formed via halogenation (see Syn. Comm. 1995, 25(21) 3427-3434).

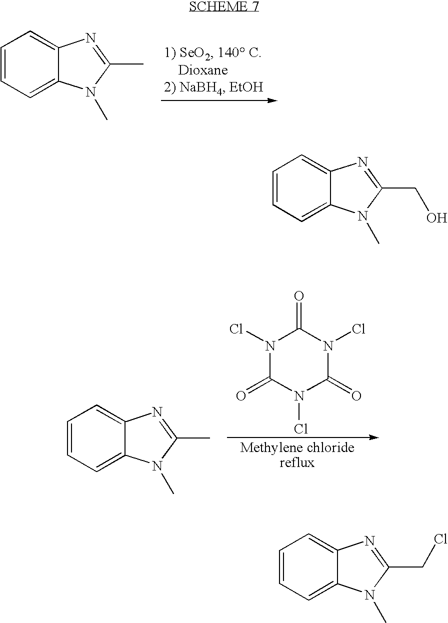

Triazole analogues can be prepared in a multitude of ways. One way is depicted in Scheme 8. Treatment of a hydrazide with dimethyl formamide dimethyl acetal to form an intermediate, which is subsequently treated with an amine or aniline with the addition of heat and acetic acid provides the 1,2,4 triazoles (see Org. Lett, 2004, 6(17), 2969-2971). The regioisomeric triazoles can be prepared by swapping the functionality of the starting materials.

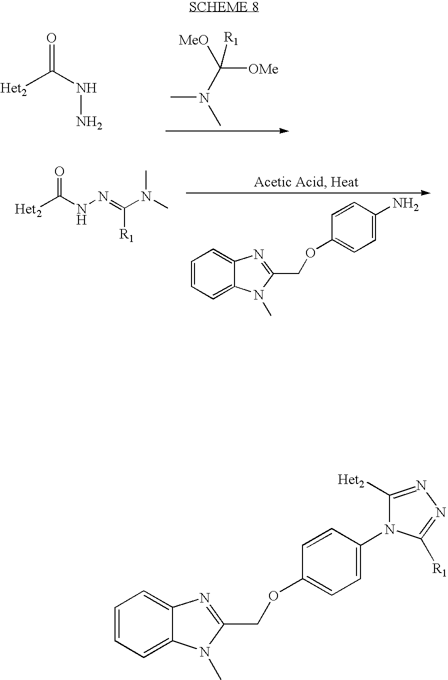

Other triazole isomers can be prepared according to scheme 9 by starting with the carboxyamides and treating with dimethyl formamide dimethyl acetal followed by the addition of aromatic hydrazines. The regioisomeric triazoles can be prepared by swapping the functionality of the starting materials.

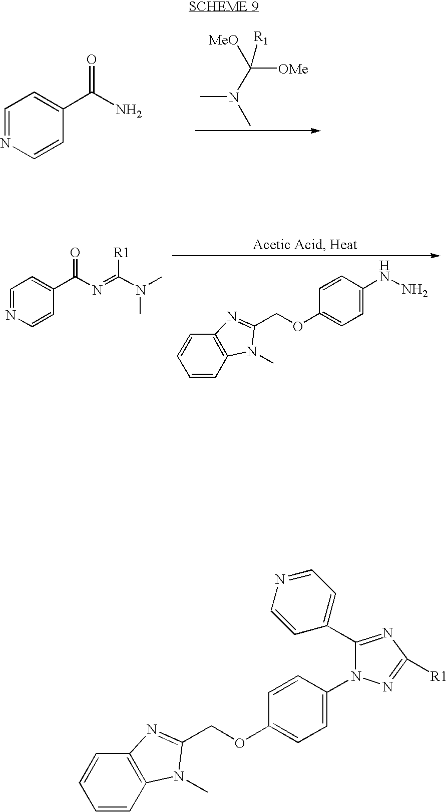

The inverted ketone isomer can be prepared according to Scheme 10. (Bunting et al. JACS, 1988, 110, 4008.) The starting aldehyde is coupled with a phosphonate to provide the enaminone. The enaminone is hydrolyzed to provide the desired ketone. The ketone can then be utilized according to Scheme 1,2 and 3 to provide the desired compounds.

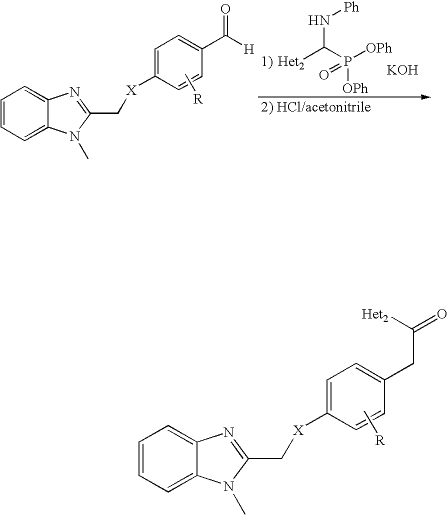

Step 1 of Scheme 11 is an imine formation/heterocycle formation. A compound of formula 2 wherein R1 is alkyl, benzyl, or allyl, is condensed with 4 pyridine carboxaldehyde in solvent such as toluene and is heated to reflux with a Dean-Stark apparatus attached to remove water for about 40 hours. After removal of toluene, the crude imine was mixed with tosylmethylisocyanide and a base such as potassium carbonate, in a solvent mixture of 1,2 -dimethoxyethane and methanol, and was heated at reflux for about 3 hours to afford 3A.

Step 2 of Scheme 11 is a phenol dealkylation. If R1 is methyl, the dealkylation can be effected with boron tribromide (BBr3) in a non-coordinating solvent such as methylene chloride at about 20-40° C. for about 3-48 hours, where about 24 hours is preferred to yield 4A. If R2 is benzyl, the dealkylation can be effected with in neat trifluoracetic acid with anisole at a temperature of about 75° C. for about 3-48 hours, where about 24 hours is preferred to yield 4A. If R1 is allyl, the dealkylation can be effected with a palladium catalyst, such as dichloropalladium bis(triphenylphosphine) of palladium acetate, where dichloropalladium bis(triphenylphosphine) is preferred, with a reducing agent such as n-butylammonium formate, in a solvent such as tetrahydrofuran, 1,2-dichloroethane, methylene chloride, or an alkanol, where 1,2-dichloroethane is preferred, in a temperature range from about 20° C. to 75° C., to yield 4A.

Step 3 of Scheme 11 is a phenol alkylation. Treatment of 4A with a base such as potassium carbonate, sodium carbonate, cesium carbonate, sodium hydride, or potassium hydride, where cesium carbonate or sodium hydride are preferred, an alkylating agent such as Cl—CH₂Het³, in a solvent such as tetrahydrofuran, 1,2-dimethoxyethane, N,N-dimethylformamide, dimethylacetamide, N-methylpyrrolidinone, or dimethylsulfoxide, where dimethylsulfoxide or N,N-dimethylformamide are preferred, at a temperature from about 20° C. to 70° C., where about 23° C. is preferred, for about 3-48 hours, where about 24 hours is preferred, affords 1A.

Step 4 of Scheme 11 is an imidazole deprotonation/electrophilic trapping. Treatment of 3A with a base such as lithium diisopropyl amide or lithium 2,2,6,6-tetramethylpiperidine, where lithium diisopropylamide is preferred, in a solvent such as tetrahydrofuran, at a temperature from about −78° C. to −20° C., where about -20° C. is preferred, for about 5 minutes to 30 minutes, where about 10 minutes is preferred, followed by addition of the desired electrophile R3-I, affords 3B.

Step 5 of Scheme 11 is a phenol dealkylation and uses the same methods as described for Step 2 above to produce 4B.

Step 6 of Scheme 11 is a phenol alkylation and uses the same methods as described for Step 3 above to produce 1B.

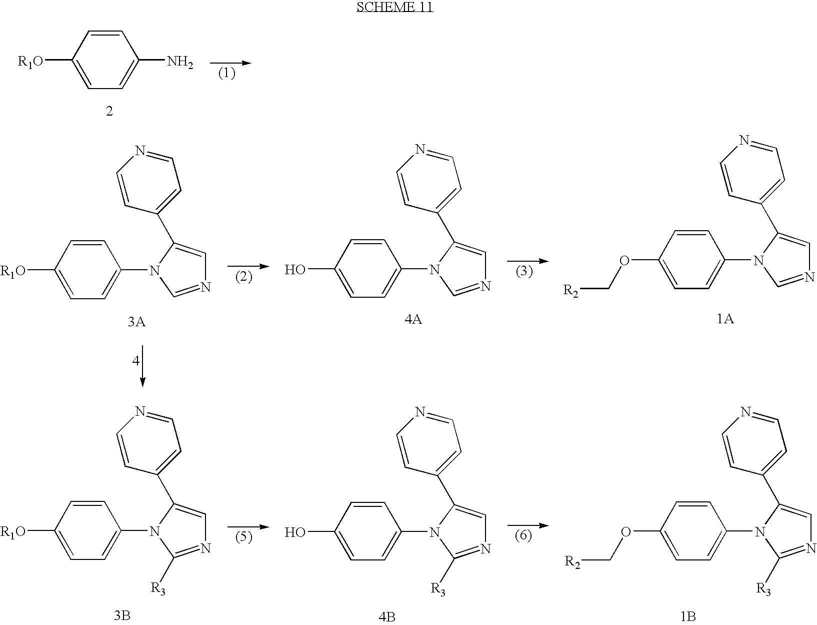

Step 1 of Scheme 12 is an acylation of an amine to form an amide. Compound 2, wherein R1 can be methyl, benzyl, or allyl, is treated with an acid chloride or a carboxylic acid in the presence of a coupling reagent, such as tri-n-propylphosphonic anhydride or dicyclohexyl carbodiimide, where tri-n-propylphosphonic anhydride is preferred, in the presence of a base such as sodium hydroxide, potassium or sodium carbonate, trimethylamine, or diisopropylethylamine, where diisopropylethylamine is preferred, in a solvent system such as water/methylene chloride, water/ethyl acetate, ethyl acetate, tetrahydrofuran, or methylene chloride, where ethyl acetate is preferred, at a temperature from about 0° C. to 50° C., where about 20° C. to 30° C. is preferred, to yield 5.

Step 2 consists of a chlorination to form an iminochloride, reaction with an amine to form an amidine, followed by treatment with acid to form an imidazole. Compound 5 is treated with a chlorinating agent such as PCl₅/POCl₃ at a temperature of about 120° C. for about 4 hours. The chlorinating agent is removed in vacuo and an excess of 1,1-diethoxy-2-ethylamine in a solvent such as isopropanol is added and the mixture is stirred for about 5-24 hours at about 23° C. The solvent is removed in vacuo and concentrated hydrochloric acid and isopropanol is added and the mixture is heated to about 90° C. for about 24 hours to yield 6.

Step 3 of Scheme 12 is a phenol dealkylation. If R1 is methyl, the dealkylation can be effected with boron tribromide (BBr3) in a non-coordinating solvent such as methylene chloride at about 20-40° C. for about 3-48 hours, where about 24 hours is preferred to yield 7. If R2 is benzyl, the dealkylation can be effected with in neat trifluoracetic acid with anisole at a temperature of about 75° C. for about 3-48 hours, where about 24 hours is preferred to yield 7. If R1 is allyl, the dealkylation can be effected with a palladium catalyst, such as dichloropalladium bis(triphenylphosphine) of palladium acetate, where dichloropalladium bis(triphenylphosphine) is preferred, with a reducing agent such as n-butylammonium formate, in a solvent such as tetrahydrofuran, 1,2-dichloroethane, methylene chloride, or an alkanol, where 1,2-dichloroethane is preferred, in a temperature range from about 20° C. to 75° C., to yield 7.

Step 4 of Scheme 12 is a phenol alkylation. Treatment of 7 with a base such as potassium carbonate, sodium carbonate, cesium carbonate, sodium hydride, or potassium hydride, where cesium carbonate is preferred, in a solvent such as tetrahydrofuran, 1,2-dimethoxyethane. N,N-dimethylformamide, dimethylacetamide, N-methylpyrrolidinone, or dimethylsulfoxide, where dimethylsulfoxide is preferred, at a temperature from about 20° C. to 70° C., where about 23° C. is preferred, for about 3-48 hours, where about 24 hours is preferred, affords 1C.

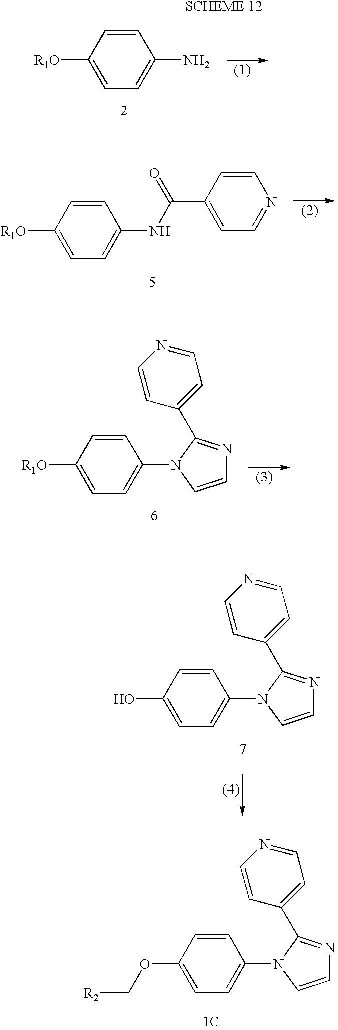

The following Examples illustrate the present invention. It is to be understood, however, that the invention, as fully described herein and as recited in the claims, is not intended to be limited by the details of the following Examples.

### Experimental Procedures

Preparation 1

4-benzyloxy-N-methoxyl-N-methyl-benzamide

To a solution of 4-Benzyloxy-benzoic acid (46.17 g) in dioxane (500 ml)/acetonitrile (500 ml) was added triethyl amine (38 ml) and O,N-Dimethyl-hydroxylamine hydrogen chloride (28 g) and the reaction mixture stirred at ambient temperature from 24 hours. The reaction mixture was filtered (triethyl amine hydrogen chloride) and concentrated. The reaction mixture was dissolved in methylene chloride and washed with water, dried magnesium sulfate, filtered and concentrated to provide the title compound (54 g). MS: (M⁺H m/z=272.3)

Preparation 2

1-(4-Benzyloxy-phenyl)-2-pyridin-4-yl-ethanone

To a solution of Lithium diisopropyl amide (1.0 M, 3 eq.) in tetrahydrofuran was added 4-picoline dropwise (12.8 ml, 3 eq.) at 0° C. under N₂. After 30 min the anion was cooled to −78° C. In a separate round bottom flask 4-benzyloxy-N-methoxy-N-methylbenzamide (11.9 g, 44 mmole) was dissolved in tetrahydrofuran (132 ml) and cooled to −78° C. under N₂. 1.2 eq. of the 4-picoline anion was added dropwise to the amide solution. After 45 min, 1 eq. more of the 4-picoline anion was added. After an addition 30 min, acetic acid (10 ml) was added dropwise and the reaction was slowly warned to ambient temperature. The pH was adjusted to 9 with saturated sodium bicarbonate, diluted with water and extracted 3× methylene chloride. The organic layer was dried magnesium sulfate filtered and concentrated to provide the title compound (9.5 g, 73%). MS: (M⁺H m/z=304.2)

Preparation 3

4-[3-(4-Benzyloxy-phenyl)-1-methyl-1H-pyrazol-4-yl]-pyridine

To a solution of 1-(4-Benzyloxy-phenyl)-2pyridin-4-yl-ethanone (26.2 g) in toluene (175 ml) was added diethoxymethyl-dimethyl-amine (16.3 ml) and the reaction mixture was heated at reflux for 1 h. The reaction mixture was cooled to ambient temperature and methyl hydrazine (5.1 ml) was added. The reaction was complete in two hours and concentrated. The solid was triturated with ethyl acetate and filtered to provide the title compound (isomer ratio 14:1). MS: (M⁺H m/z 342.2)

Preparation 4

4-(1-Methyl-4-pyridin-4-yl-1-H-pyrazol-3-yl)-phenol

To a solution of 4-[3-(4-Benzyloxy-phenyl)-1-methyl-1H-pyrazol-4-yl]-pyridine (1.28 g) in ethanol (50 ml)/ethyl acetate (5 ml) in a parr bottle was added Palladium hydroxide (500 mg). The parr bottle was charged to 40 psi on a shaker for 6 h. The reaction mixture was filtered and concentrated. MPLC biotage chromatography eluting with methanol (1-7%)/chloroform provided the title compound (860 mg, 91%). ¹H NMR (400 MHz, DMSO) δ 9.53 (5, 1H), 8.39 (d, J=5.8 Hz, 2 H), 7.15 (m, 4H), 6.72 (d, J=8.7 Hz, 1H), 3.84 (S, 3H); MS: (M⁺H m/z=252.2)

Preparation 5

4-[3-(4-Benzyloxy-phenyl)-1H-pyrazol-4-yl]-pyridine

To a solution of 1-(4-Benzyloxy-phenyl)-2-pyridin-4-yl-ethanone (1.58 9) was added toluene (26 ml) and 1.6 g of Diethoxymethyl-dimethyl-amine and the reaction mixture heated at reflux for 1 h. The reaction mixture was concentrated, dissolved in methanol (26 ml) and hydrazine (0.64 g) and the reaction mixture was heated at reflux for 1h. The reaction mixture was concentrated and purified via biotage MPLC eluting with 5% methanol/chloroform/0.5% ammonium hydroxide to provided the title compound (0.89 g). MS: (M⁺H m/z=328.1)

Preparation 6

4-[3-(4-Benzyloxy-phenyl)-1-(2,2,2-trifluoro-ethyl)-1H-pyrazol-4yl]-pyridine

To a solution of 4-[3-(4-Benzyloxy-phenyl)-1H-pyrazol-4-yl]-pyridine (0.42 g) in dimethyl formamide (7 ml) was added cesium carbonate (0.65 g) and 1,1,1-Trifluoro-2-iodo-ethane (0.29 ml). The reaction mixture was heated at 60° C. for 24 h, poured into water and extracted 3× with dichloromethane. Purification via biotage MPLC chromatography, eluting with 5% methanol/0.5% ammonium hydroxide/70% ethyl acetate/hexane provided the title compound. MS: (M⁺H m/z=410.0)

Preparation 7

4-[4-Pyridin-4-yl-1-(2,2,2-trifluoro-ethyl)-1-H-pyrazol-3-yl]-phenol

Following the procedure for the preparation of 4-(1-Methyl-4-pyridin-4-yl-1H -pyrazol-3-yl)-phenol but substituting 4-[3-(4-Benzyloxy-phenyl)-1-(2,2,2-trifluoro-ethyl)-1H-pyrazol-4-yl]-pyridine provided the title compound. MS: (M⁺H m/z=320.1)

Preparation 8

4-(1-Methyl-1H-benzoimidazol-2-ylmethoxy)-benzoic acid methyl ester

To a solution of 2-Chloromethyl-1-methyl-1H-benzoimidazole (5 g) in acetone (150 mL) was added potassium carbonate (8.6 g) and 4-Hydroxy-benzoic acid methyl ester (3.84 g) and the reaction mixture heated at reflux for 24 h. The reaction mixture was concentrated, dissolved in methylene chloride and washed with 1N NaOH, dried magnesium sulfate, filtered and concentrated to give 7.4 g. MS: (M⁺H m/z=297.2)

Preparation 9

4-(1-Methyl-1H-benzoimidazol-2-ylmethoxy)-benzoic acid

To a solution of 4-(1Methyl-1H -benzoimidazol-2-ylmethoxy)-benzoic acid methyl ester (7.4 g) in tetrahydrofuran (125 mL) and methanol (40 mL) was added 1N NaOH and the reaction stirred for 18 h. The reaction pH was adjusted to 1 with 1N HCl and the precipitate was filtered and dried in a vac oven to give 5.73 g of a white solid. MS, (M⁺H m/z=283.2)

Preparation 10

N-Methoxy-N-methyl-4-(1-methyl-1H-benzoimidazol-2-ylmethoxy)-benzamide

Following the procedure for the preparation of 4-benzyloxy-N-methoxy-N-methyl-benzamide but substituting 4-(1-Methyl-1H-benzoimidazol-2-ylmethoxy)-benzoic acid provided the title compound. MS: (M⁺H m/z=326.2)

Preparation 11

1-[4-(1-Methyl-1H-benzoimidazol-2-ylmethoxy)-phenyl]-2-pyridin-4-yl-ethanone

Following the procedure for the preparation of 1-(4-Benzyloxypheny)-2-pyridin-4-yl-ethanone but substituting N-Methoxy-N-methyl-4-(1methyl-1H-benzoimidazol-2-ylmethoxy)-benzamide provided the title compound. MS: (M⁺H m/z=355.2)

### EXAMPLE 1

1-Methyl-2-[4-(4-pyridin-4-yl-1H-pyrazol-3-yl)-phenoxymethyl]-1H-benzoimidazole

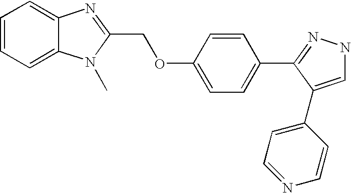

Following the procedure for the preparation of 4-[3(4-Benzyloxy-phenyl)-1H-pyrazol-4-yl]-pyridine but substituting 1-[4-(1-Methyl-1H-benzoimidazol-2-ylmethoxy)-phenyl]-2-pyridin-4-yl-ethanone provided the title compound (65%). ¹H NMR (400 MHz, CD₃OD) δ8.44 (d J=6.2 Hz, 2 H), 7.66 (d, J=7.9 Hz, 1 H), 7.55 (d, J=7.9 Hz, 1H), 7.37-7,30 (m, 7 H), 7.18 (m, 2H), 5.45 (s, 2H), 3.93 (s, 3H), MS: (M⁺H m/z=382.1), PDE10 IC₅₀=079 nm.

### EXAMPLE 2

2-[4-(1-Ethyl-4-pyridin-4-yl-1-H-pyrazol-3-yl)-phenoxylmethyl]-1-methyl-1-1H-benzoimidazole

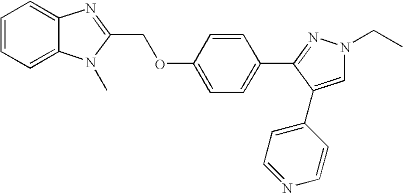

Following the procedure for the preparation of 4-(1-Methyl-4-pyridin-4-yl-1H-pyrazol-3-yl)-phenol but substituting ethyl hydrazine and 1-[4-(1-Methyl-1-H-benzoimidazol-2-ylmethoxy)-phenyl]-2-pyridin-4-yl-ethanone provided the title compound. MS: (M⁺H m/z=410.2): PDE10 IC₅₀=1.38 nm.

### EXAMPLE 3

1-{3-[4(1-Methyl-1H-benzoimidazol-2-ylmethoxy)-phenyl]-4-pyridin-4-yl-pyrazol-1-yl)-propan-2-ol

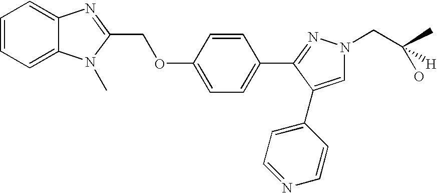

Following the procedure for the preparation of 2-[4-(1-Ethyl-4-pyridin-4-yl-1-H-pyrazol-3-yl)-phenoxymethyl]-1-methyl-1-H-benzoimidazole but substituting (R)-1-Hydrazino-propan-2-ol provided the title compound. MS: (M⁺H m/z=440.2); PDE10 IC₅₀=2.6 nm.

### EXAMPLE 4

1-Methyl-2-[4-(4-pyridin-4-yl-isoxazol-5-yl)-phenoxymethyl]-1-H-benzoimidazole

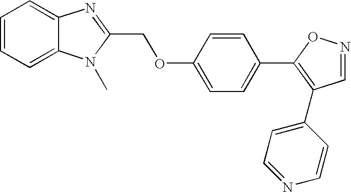

To 1-[4-(1-Methyl-1H-benzoimidazol-2-ylmethoxy)-phenyl]-2-pyridin-4-yl-ethanone (150 mg) in a flask was added diethoxytriethyl-dimethyl-amine (1 mL) were heated at reflux for 1 h and concentrated. Hydroxyl amine hydrogen chloride (32 mg). sodium bicarbonate (22 mg), acetic acid (0.02 mL), methanol (3 mL) and water (1 mL) were added and the reaction mixture heated at reflux for 1 h. The reaction mixture was poured into saturated sodium bicarbonate extracted with methylene chloride, dried magnesium sulfate filtered and concentrated. Purification with biotage MPLC eluting with 2% methanol/0.5% ammonium hydroxide/60% ethyl acetate/hexanes provided the title compound (134 mg), ¹H NMR (400 MHz, CD₃OD) δ 8.69 (s, 1H), 8,50 (d, J=4.6Hz, 2 H), 7.66 (d, J=7.9 Hz, 1 H), 7.58 (d, J=9.1 Hz, 1H), 7.53 (d, J=7.9 Hz, 1 H), 7.46 (d, J=6.2 Hz, 2H), 7.35 (m, 2H), 7.22 (d, J=9.1 Hz, 2H), 5.46 (s, 2H), 3.92 (s, 3H); MS: (M⁺H m/z=383.1); PDE10 IC₅₀=4.94 nm.

### EXAMPLE 5

1-Methyl-2-[4-(1-methyl-4-pyridin-4-yl-1H-pyrazol-3-yl)-phenoxymethyl]-1-H-benzoimidazole

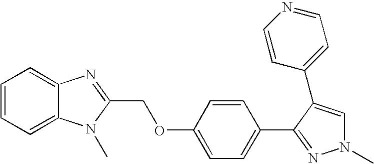

To a solution of 4-(1-Methyl-4-pyridin-yl-pyrazol-3-yl)-phenol (50 mg) in dioxane (1 ml) was added triphenyl phosphine (83 mg), (1-Methyl-1-H-benzoimidazol-2-yl)-methanol (48 mg) and Di-t-butyl azodicarboxylate (73 mg). The reaction mixture was heated at 60° C. for 18, poured into 1 N NaOH, extracted 3× with chloroform, dried magnesium sulfate, filtered and concentrated. Purification via biotage MPLC eluting with 80% ethyl acetate/hexane provided the title compound (75 mg, 96%)). ¹H NMR (400 MHz, CCDl₃) δ 8.44 (d, J=6.2 Hz, 2 H), 7.76 (dd, J=7.1, 1.7 Hz, 1 H), 7.55 (s, 1H), 7.37-7.28 (m, 5 H), 7.15 (dd, J=4.6, 1.7 Hz, 2H), 7.05 (d, J=9.1 Hz, 2H), 5.38 (s, 2H), 3.94 (s, 3H) 3.88 (s, 3H); MS: (M⁺H m/z=396.2); PDE10 IC₅₀=0.56 nm.

### EXAMPLE 6

1-Methyl-2-[4-(2 methyl-4-pyridin-4-yl-2H-pryazol-3-yl)-phenoxymethyl]-1H-benzoimidazole

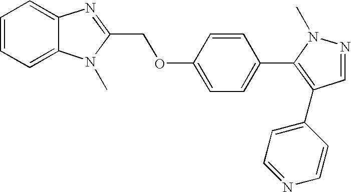

Following the procedure for the preparation of 1-Methyl-2-[4-(-1-methyl-4-pyridin-4-yl-1H-pyrazol-3-yl)-phenoxymethyl]-1H-benzoimidazole but substituting 4-(2-Methyl-4-pyridin-4-yl-2H-pyrazol-3-yl)-phenol (minor isomer from the preparation of 4-[3-(4-Benzyloxy-phenyl)-1-methyl-1H-pyrazol-4-yl]-pyridine) provided the title compound. ¹H NMR (400 MHz, CDCl₃) δ 8.36 (d, J=6.2 Hz, 2 H), 7.78 (s, 1H), 7.78 (d, J=5.4 Hz, 1H), 7.38 (m, 2H), 7.19 (m, 5 H), 7.01 (d, J=6.2 Hz, 2H), 5.43 (s, 2H). 3.92 (s, 3H) 3.70 (s, 3H); MS: (M⁺H m/z=396.2); PDE10 IC₅₀=1.84 nm.

### EXAMPLE 7

1-Fluoromethyl-2-[4-(1-methyl-4-pyridin-4-yl-1H-pyrazol-3-yl)-phenoxymethyl]-1H-benzoimidazole

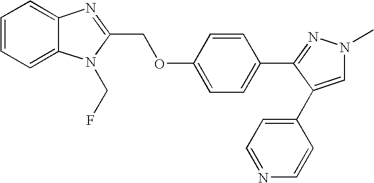

Following the procedure for the preparation of 1-Methyl-2-[4-(1methyl-4-pyridin-4-yl-1H-pyrazol-3-yl)-phenoxymethyl]-1H-benzoimidazole but substituting (1-Fluoromethyl-1H-benzoimidazol-2-yl)-methanol provided the title compound. ¹NMR (400 MHz, CDCl₃) δ 8.45 (d, J=6.2 Hz, 2H), 7.79 (d, J=7.5 Hz, 1H), 7.55 (s, 1H), 7.48 (d, J=7.9 Hz, 1H), 7.39 (m, 4H), 7.13 (d, J=6.2 Hz, 2H), 7.04 (d, J=9.1 Hz, 2H), 6.37 (s, 1H), 6.24(s, 1H), 5.45 (s, 2H), 3.94 (s, 3H), MS: (M⁺H m/z=414.2), PDE10 IC₅₀=0.98 nm.

### EXAMPLE 8

1-Isopropyl-2-[4-(1-methyl-4-pyridin-4-yl-1H-pyrazol-3-yl)-phenoxymethyl]-1H-benzoimidazole

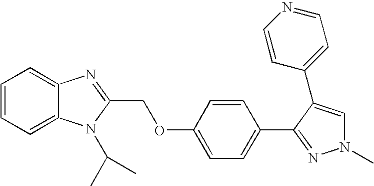

Following the procedure for the preparation of 1-Methyl-2-(4-(1-methyl-4-pyridin-4-yl-1H-pyrazol-3-yl-phenoxymethyl-1H-benzoimidazole but substituting (1-isopropyl-1H-benzoimidazol-2-yl)-methanol provided the title compound. ¹H NMR (400 MHz, CDCl₃) δ 8.44 (d, J=5.8 Hz, 2H), 7.76 (m, 1 H), 7.56 (s, 1H), 7.54 (m, 2 H), 7.37 (d, J=8.7 Hz, 2H), 7.25 (m, 2H), 7.15 (d, J=6.2 Hz, 2H), 7.04 (d, J=8.7 Hz, 2H), 5.37 (s, 2H), 4.94 (m, 1H) 3.95 (s, 3H), 1.65 (d, J=7.1 Hz, 6H); MS: (M⁺H m/z=424.1); PDE10 IC₅₀=8.91 nm.

### EXAMPLE 9

1-Cyclopropyl-2-[4-(1-methyl-4-pyridin-4-yl-1H-pyrazol-3-yl)-phenoxymethyl]-1-H-benzoimidazole

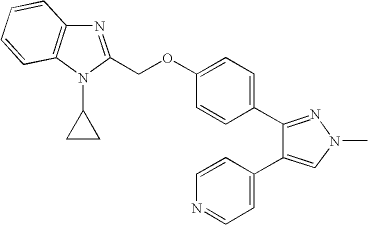

Following the procedure for the preparation of 1-Methyl-2-[4-(1-methyl-4-pyridin-4-yl-1H-pyrazol-3-yl)-phenoxymethyl]-1H-benzoimidazole but substituting (1-Cyclopropyl-1H-benzoimidazol-2-yl)-methanol provided the title compound. ¹H NMR (400 MHz, CDCl₃) δ 8.43 (d, J=6.2 Hz, 2H), 7.72 (d, J=6.6 Hz, 1H), 7.54 (s, 1H), 7,53 (d, J=6.2 Hz, 1H), 7.37 (d, J=9.17 Hz, 2H), 7.24 (m, 2H), 7.13 (d, J=6.2 Hz, 2H), 7.01 (d, J=9.1 Hz, 2H), 5.37 (s, 2H), 3.92 (s, 3H), 3.41 (m, 1H), 1.21 (m, 4H); MS: (M⁺H m/z=422.1); PDE10 IC₅₀=0.69 nm.

### EXAMPLE 10

1-(2-Methoxy-ethyl)-2[4-(1-methyl-4-pyridin-4-yl-1H-pyrazol-3-yl)-phenoxymethyl]-1H-benzoimidazole

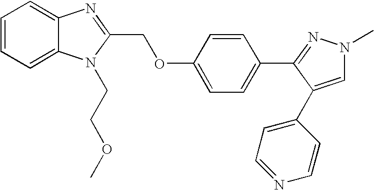

Following the procedure for the preparation of 1-Methyl-2-[4-(1-methyl-4-pyridin-4-yl-1H-pyrazol-3-yl)-phenoxymethyl]-1H-benzoimidazole but substituting [1-(2-Methoxy-ethyl)-1H-benzoimidazol-2-yl]-methanol provided the title compound. ¹H NMR (400 MHz, CDCl₃) δ 8.45 (d, J=6.2 Hz, 2H), 7.75 (m, 1 H), 7.54 (s, 1 H), 7.37 (m, 3H), 7.26 (m, 2H). 7.13 (d, J=6.2 Hz, 2H), 7.04 (d, J=8.7 Hz, 2H), 5.42 (s, 2H), 4.48 (t, J=5.4 Hz, 1H), 3.93 (s, 3H). 3.71 (t, J=5.8 Hz, 2H), 3.23 (s, 3H); MS: (M⁺H m/z=440.2); PDE10 IC₅₀=1.6 nm.

### EXAMPLE 11

2-[4-(1-Methyl-4-pyridin-4yl-1H-pyrazol-3-yl)-phenoxymethyl]-imidazo[1,2-a]pyridine

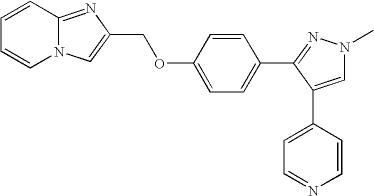

Following the procedure for the preparation of 1-Methyl-2-[4-(1-methyl-4-pyridin-4-yl-1H-pyrazol-3-yl)phenoxymethyl]-1H-benzoimidazole but substituting Imidazo[1,2-a]pyridin-2-yl-methanol provided the title compound. ¹H NMR (400 MHz, CDCl₃) δ 8.48 (d, J=6.2, 2 H), 7.87 (d, J=7.1 Hz, 1H), 7.60 (d, J=9.1 Hz, 1H), 7.57 (s, 1H), 7.38 (t, J=8.7 Hz, 2 H), 7.18 (d, J=6.2 Hz, 2H), 7.04 (d, J=8.7 Hz, 2H): 6.86 (d, J=6.6 Hz, 1H), 5.28 (s, 2H): 3.97 (s, 3H) 2.52 (s, 3H); MS: (M⁺H m/z=382.1); PDE10 IC₅₀=0.53 nm.

### EXAMPLE 12

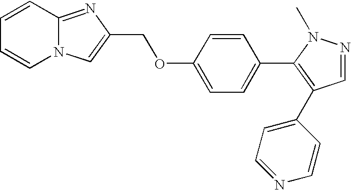

Following the procedure for the preparation of 1-Methyl-2-[4-(1-methyl-4-pyridin-4-yl-1H-pyrazol-3-yl)-phenoxymethyl]-1H-benzoimidazole but substituting 4-(2-Methyl-4-pyridin-4-yl-2H-pyrazol-3-yl)-phenol (minor isomer from the preparation of 4-[3-(4-Benzyloxy-phenyl)-1-methyl-1H-pyrazol-4-yl]pyridine) and imidazo[1,2-a]pyridin-2-yl-methanol provided the title compound. ¹H NMR (400 MHz, CDCl₃) δ 8.45 (d: J=6.2 Hz, 2H), 8.06 (d, J=6.6 Hz, 1 H), 7.61 (s, 1H) 7.55 (s, 1 H), 7.37 (d, J=8.7 Hz, 2H), 7.15 (m: 4H): 6.99 (d: J=8.7 Hz, 2H): 6.77 (t, J=5.7 Hz, 1H): 5.27 (s, 2H): 3.93 (s, 3H); MS: (M⁺H m/z=382.1); PDE10 IC₅₀=0.53 nm.

### EXAMPLE 13

2-[4-(1-Methyl-4-pyridin-4-yl-1H-pyrazol-3-yl)-phenoxymethyl]-[1,2,4]triazolo[1,5-a]-pyridine

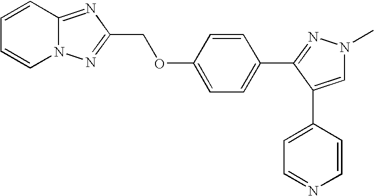

Following the procedure for the preparation of 1-Methyl-2-[4-(1-methyl-4-pyridin-4-yl-1H-pyrazol-3-yl)-phenoxymethyl]-1H-benzoimidazole but substituting [1,2,4]Triazolo[1,5a]pyridin-2-yl-methanol provided the title compound. 8.57 (d, J=6.6 Hz, 1H), 8.44 (d, J=5.8 Hz, 2H), 7.73 (d, J=9.1 Hz, 1H), 7.55 (s, 1H), 7.54 (dd, J=6.6, 1.3 Hz, 1 H), 7.38 (d, J=8.7 Hz, 2H), 7.14 (d, J=5.8 Hz, 2H), 7.05 (m, 3H), 5.36 (s, 2H), 3.94 (s. 3H); MS: (M⁺H m/z=383.1); PDE10 IC₅₀=1.14 nm.

### EXAMPLE 14

2-{4-[4-Pyridin-4-yl-1-(2,2,2-trifluoro-ethyl)-1-H-pyrazol-3-yl]-phenoxymethyl}-[1,2,4]triazolo[1,5-a]pyridine

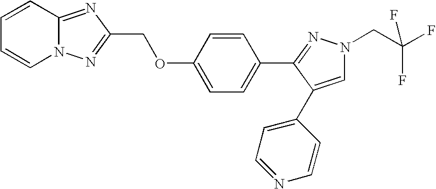

Following the procedure for the preparation of 1-Methyl-2-[4-(2-methyl-4-pyridin-4-yl-2H-pyrazol-3-yl)-phenoxymethyl]-1H-benzoimidazole but substituting 4-[4-Pyridin-4-yl-1-(2,2,2-trifluoro-ethyl)-1H-pyrazol-3-yl]-phenol and [1,2,4]Triazolo[1,5-a]pyridin-2-yl-methanol provided the title compound. ¹H NMR (400 MHz, CDCl₃) δ8.57 (d, J=7.1 Hz, 1 H), 8.48 (d, J=6.2 Hz, 2H), 7.73(d. J=8.7 Hz, 1H), 7.68 (s 1H), 7.53 (t, J=75 Hz, 1 H), 7.37 (d, J=7.7 Hz., 2H), 7.16 (dd, J=6,2, 1.7 Hz, 2H), 7.03 (m, 3H), 5.39 (s, 2H), 4.77 (q, J=8.3 Hz, 2H); MS: (M⁺H m/z=451.0); PDE10 IC₅₀=0.56 nm.

### EXAMPLE 15

2-{4-[4-Pyridin-4-yl-1-(2,2,2-trifluoro-ethyl-1H-pyrazol-3-yl]-phenoxymethyl}-imidazo[1,2-a]pyridine

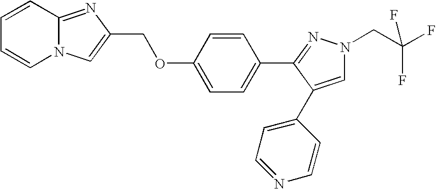

Following the procedure for the preparation of 1-Methyl-2[4-(2-methyl-4-pyridin-4-yl-2H-pyrazol-3-yl)-phenoxymethyl-]-1H-benzoimidazole but substituting 4-[4-Pyridin-4-yl-1-(2,2,2-trifluoro-ethyl)-1H-pyrazol-3-yl]-phenol and Imidazo[1,2-a]pyridin-2-yl-methanol provided the title compound, ¹H NMR (400 MHz. CDCl₃) δ 8.50 (d, J=5.8 Hz, 2H), 8.08 (d, J=7.1 Hz, 1H), 7.69 (s, 1H), 7.62 (s, 1H), 7.60(d, J=9.1 Hz, 1H), 7.38 (d, J=8.7 Hz, 2H), 7.18 (m, 3H), 7.01 (d, J=8.7 Hz, 2H), 6.79 (t, J=6.6 Hz, 1H), 5.29 (s, 2H), 4.77 (q, J=8.3 Hz, 2H); MS: (M⁺H m/z=450.2); PDE10 IC₅₀=0.39 nm.

### EXAMPLE 16

1-Methyl-2-{4-[4-pyridin-4-yl-1-(2,2,2-trifluoro-ethyl)-1H-pyrazol-3yl]-phenoxymethyl}-1H-benzoimidazole

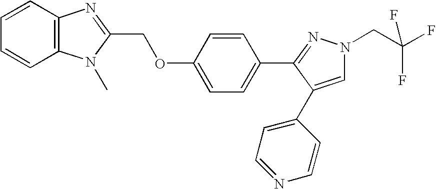

Following the procedure for the preparation of 1-Methyl-2-[4-(2-methyl-4-pyridin-4-yl-2H-pyrazol-3-yl)-phenoxymethyl]-1H-benzoimidazole but substituting 4-[4-Pyridin-4-yl-1-(2,2,2-trifluoro-ethyl)-1H-pyrazol-3-yl]-phenol provided the title compound. ¹H NMR (400 MHz, CDCl₃) δ 8.49 (d, J=6.2 Hz, 2H), 7.77 (d, J=7.5 Hz, 1H), 7.67 (s, 1H), 7.38 (d, J=9.1 Hz, 2H), 7.33 (m, 3H), 7.16 (d, J=6.3 Hz, 2H), 7.06 (d, J=8.7 Hz, 2H). 5.39 (s, 2H). 4.77 (q, J=8.3 Hz, 2H) 3.88 (s, 3H); MS: (M⁺H m/z=464.2); PDE10 IC₅₀=0.21 nm.

### EXAMPLE 17

1-Fluoromethyl-2-{4-[4-pyridin-4-yl-1-(2.2.2-trifluoro-ethyl)-1-H-pyrazol-3-yl]-phenoxymethyl}-1H-benzoimidazole

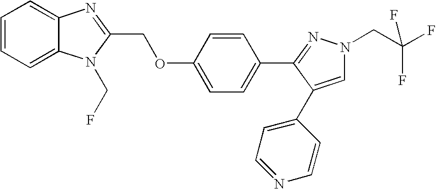

Following the procedure for the preparation of 1-Methyl-2[4-(2-methyl-4-pyridin-4-yl-2H-pyrazol-3-yl)-phenoxymethyl]-1H-benzoimidazole but substituting 4-[4-Pyridin-4-yl-1-(2,2,2-trifluoro-ethyl)-1H-pyrazol-3-yl]-phenol and (1-Fluoromethyl-1H-benzoimidazol-2-yl)-methanol provided the title compound. ¹H NMR (400 MHz, CDCl₃) δ 8.50 (d, J=5.8 Hz, 2H), 7.79 (d, J=7.5 Hz, 1H), 7.67 (s, 1H), 7.46 (m, 1H), 7.40 (m, 4H), 7.16 (4, J=6.2 Hz 2H), 7.05 (d, J=9.1 Hz, 2H), 6.38 (s, 1H), 6.24 (s, 1H), 5.46 (s, 2H), 4.12 (q, J=7.1 Hz, 2H); MS: (M⁺H m/z=481.9). PDE10 IC₅₀=0.75 nm.

### EXAMPLE 18

1-Methyl-2-[4-(1-methyl-4-pyridin-4-yl-1H-pyrazol-3yl)-phenoxymethyl-1-H-imidazo[4,5b]pyridine

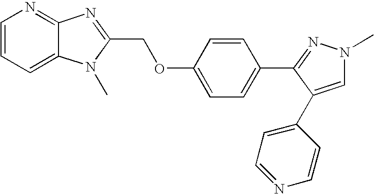

Following the procedure for the preparation of 1-Methyl-2-[4-(1-methyl-4-pyridin-4-yl-1H-pyrazol-3-yl)-phenoxymethyl-1H-benzoimidazole but substituting (1-Methyl-1-H-imidazo[4,5-b]pyridin-2-yl)-methanol provided the title compound. ¹H NMR (400 MHz, CDCl₃) δ 8.52 (d, J=4.6 Hz, 1 H), 8.43 (m, 2H), 7.65 (4, J=7.9 Hz, 1H), 7.54 (s, 1H), 7.36 (d, J=8.3 Hz, 2H), 7.20 (m, 1H), 7.11(d, J=5.4 Hz, 2H), 7.04 (d, J=8.7 Hz, 2H), 5.42 (s, 2H), 3.92 (s, 3H) 3.88 (s, 3H), MS: (M⁺H m/z=397.0); PDE10 IC₅₀=9.31 nm.

### EXAMPLE 19

1-Methyl-2-[4-(1-methyl-4-pyridin-4-yl-1H-pyrazol-3-yl)-phenoxymethyl]-1-H-imidazo[4,5-c]pyridine

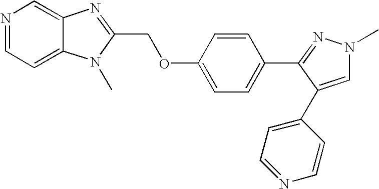

Following the procedure for the preparation of 1-Methyl-2-[4-(1-methyl-4-pyridin-4-yl-1H-pyrazol-3-yl)-phenoxymethyl]-1-H-benzoimidazole but substituting (1-Methyl-1H-imidazo[4.5-c]pyridin-2-yl)-methanol provided the title compound. ¹H NMR (400 MHz, CDCl₃) δ 9.07 (s 1H), 8.45 (m, 3H), 7.55 (s, 1H), 7.40 (d, J=8.7 Hz, 2H), 7.31 (d, J=5.5 Hz, 1 H), 7.14 (d, J=5.8 Hz, 2H), 7.03 (d, J=8.7 Hz, 2H), 5.40 (s 2H), 3.94 (s, 3H) 3.90 (s, 3H); MS: (M⁺H m/z=397.1); PDE10 IC₅₉=133 nm.

### EXAMPLE 20

5,6-Difluoro-1-methyl-2-[4-(1-methyl-4-pyridin-4-yl-1H-pyrazol-3-yl)-phenoxymethyl]-1H-benzoimidazole

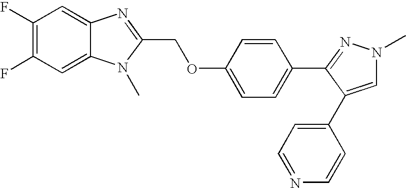

Following the procedure for the preparation of 1-Methyl-2-[4-(-1-methyl-4-pyridin-4-yl-1H-pyrazol-3-yl)-phenoxymethyl]-1H-benzoimidazole but substituting (5,6-Difluoro4-methyl-1H-benzoimidazol-2-yl)-methanol provided the title compound. ¹H NMR (400 MHz, CDCl₃ ) δ 8.46 (d, J=6.2 Hz, 2H), 7.55 (s, 1H), 7.51 (dd, J=10.3, 3.3 Hz, 1H), 7.38 (d, J=9.1 Hz, 2H), 7.14 (m, 3H), 7.02 (d, J=8.7 Hz, 2H), 5.33 (s, 2H), 3.94 (m, 3H) 3.83 (s, 3H); MS: (M⁺H m/z=432.2); PDE10 IC₅₀=16.7 nm.

### EXAMPLE 21

2-[4-(1-Methyl-4-pyridin-4-yl-1H-pyrazol-3-yl)-phenoxymethyl]-benzothiazole

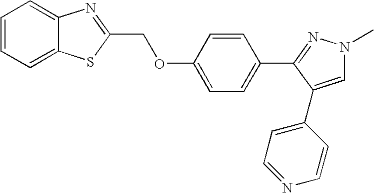

To a solution of 4-(1-Methyl-4pyridin-4-yl-1H-pyrazol-3-yl)-phenol (75 mg) in dimethyl formamide (1.5 ml) was added cesium carbonate (107 mg) and 2-Bromomethyl-benzothiazole (75 mg) and the reaction mixture was heated at 60° C. for 48 h. The reaction mixture was poured into water and extracted 3× methylene chloride, dried magnesium sulfate, filtered and concentrated. Biotage MPLC eluting with 5% methanol/1% saturated ammonium hydroxide/70% ethyl actate/hexane provided the title compound, ¹H NMR (400 MHz, CDCl₃) δ 8.46 (m, 2H), 8.02 (d, J=7.9 Hz, 1H), 7.90 (d, J=7.9 Hz, 1H), 7,56 (s, 1H) 7.50 (t, J=7.1 Hz, 1 H), 7.40 (m, 3H), 7.16 (4, J=6.3 Hz, 2H), 7.02 (d, J=6.6 Hz, 2H), 5.49 (s, 2H), 3.95 (s, 3H); MS: (M⁺H m/z=399.1); PDE10 IC₅₀=0.89 nm.

### EXAMPLE 22

2-{4-[4-Pyridin-4-yl-1-(2,2 2-trifluoro-ethyl)-1H-pyrazol-3-yl]-phenoxymethyl}-benzothiazole

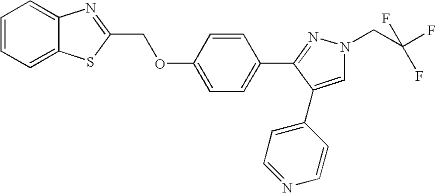

Following the procedure for the preparation of 1-Methyl-2-[4-(1-methyl-4-pyridin-4-yl-1H-pyrazol-3-yl)-phenoxymethyl]-1H-benzoimidazole but substituting 4-[4-Pyridin-4-yl-1-(2,2,2-trifluoro-ethyl)-1H-pyrazol-3-yl]-phenol provided the title compound. ¹H NMR (400 MHz, CDCl₃) δ 8.50 (d, J=6.2 Hz, 2H), 8.02 (d, J=8.3 Hz, 1H), 7.89 (d, J=7.5 Hz, 1H), 7.68 (s, 1H), 7.50 (t, J=7.1 Hz, 1H), 7.40 (m, 3H), 7.16 (d, J=6.2 Hz, 2H), 7.02 (d, J=8.7 Hz, 2H), 5.49 (s, 2H), 4.78 (q, J=8.3 Hz, 3H); MS: (M⁺H m/z=467.1); PDE10 IC₅₀=1.48 nm.

### EXAMPLE 23

2-[4-(1-Methyl-4-pyridin-4-yl-1H-pyrazol-3-yl)-phenoxymethyl]-5,6-dihydro-4H-imidazo[4,5,1-ij]quinoline

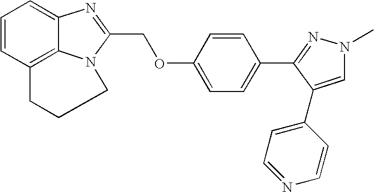

Following the procedure for the preparation of (5,6-Dihydro-4H-imidazo[4,5,1-ij]quinolin-2-yl)-methanol but substituting Benzothiazol-2-yl-methanol provided the title compound. ¹H NMR (400 MHz, CDCl₃) δ 8.45 (d, J=5.8 Hz, 2H), 7.55 (s, 1H), 7.54 (d, J=7.1 Hz, 1H), 7.37 (d, J=8.7 Hz, 2H). 7.17 (d, J=7.1 Hz, 1H), 7.14 (d, J=6.2 Hz, 2H), 7.03 (m, 3H). 5.39 (s, 2H), 4.30 (t, J=5.8 Hz, 2H), 3.94 (s, 3H), 2.98 (t, J=5.8 Hz, 2H), 2.23 (m, 2H); MS: (M⁺H m/z=422.1), PDE10 IC₅₀=3.09 nm.

### EXAMPLE 24

3-Methyl-2-[4-(1-methyl-4-pyridin-4-yl-1H-pyrazol-3-yl)-phenoxymethyl]-imidazo[1,2-a]pyridine

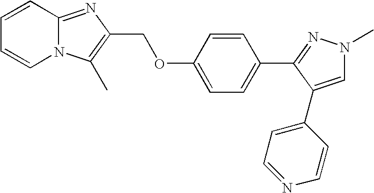

Following the procedure for the preparation of 1-Methyl-2-[4-(1-methyl-4-pyridin-4-yl-1H-pyrazol-3-yl)-phenoxymethyl]-1H-benzoimidazole but substituting (3-Methyl-imidazo[1,2-a]pyridin-2-yl)-methanol provided the title compound. ¹H NMR (400 MHz, CDCl₃) δ 8.48 (d, J=6.2, 2H), 7.87 (d, J=7,1 Hz, 1H), 7.60 (d, J=9.1 Hz, 1H), 7,57 (s, 1H), 7.38 (t J=4=8.7 Hz, 2 H), 7.18 (d, J=6.2 Hz, 2H), 7.04 (d, J=8.7 Hz, 2H), 6.86 (d, J=6.6 Hz, 1H), 5.28 (s, 2H), 3.97 (s, 3H) 2.52 (s, 3H); MS: (M⁺H m/z=396.1), PDE10 IC₅₀=1.4 nm.

### EXAMPLE 25

2-[4-(1-Methyl-4-pyridin-4-yl-1H-pyrazol-3-yl)-phenoxymethyl]-1-(2,2,2trifluoro-ethyl)-1H-benzoimidazole

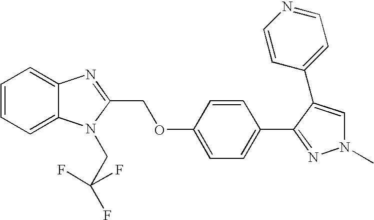

Following the procedure for the preparation of 2-[4-(1-Methyl-4-pyridin-4-yl-1H-pyrazol-3-yl)-phenoxymethyl]-benzothiazole but substituting 2-Chloromethyl-1-(2,2,2-trifluoro-ethyl)-1H-benzoimidazole provided the title compound. ¹H NMR (400 MHz CDCl₃) δ 8.46 (d, J=62, 2H), 7.80 (d, J=7.1 Hz, 1H), 7.55 (s, 1H), 7.39 (m, 5 H), 7.14 (d, J=6.2 Hz, 2H), 7.05 (d, J=9.1 Hz, 2H), 5,45 (s, 2H), 4,99 (q, J=8.3 Hz, 2H), 3.95 (s, 3H); MS: (M⁺H m/z=464.0); PDE10 IC₅₀=10 nm.

Preparation 12

3-Dimethylamino-1-pyridin-4-yl-propenone

To 1-Pyridin-4-yl-ethanone (1.62 g) was added N,N-dimethylformamide diethylacetal (10 ml) and the reaction mixture heated at 120° C. for 2 h and concentrated to provide the title compound. MS: (M⁺H m/z=177.0).

Preparation 13

4-[2-(4-Benzyloxy-phenyl-2H-pyrazol-3-yl]-pyridine

To a solution of 3-Dimethylamino-1-pyridin-4-yl-propenone (590 mg) in methanol (10 ml) was added acetic acid (0.5 ml) and (4-Benzyloxy-phenyl)-hydrazine hydrogen chloride (836 mg) and the reaction mixture heated to 60° C. for 6 h. The reaction mixture was poured into saturated sodium bicarbonate, extracted with ethyl acetate, dried magnesium sulfate, filtered and concentrated. Purification via combiflash MPLC provided the title compound (795 mg). MS: (M⁺H m/z=328.1).

Preparation 14

4-5-Pyridin-4-yl-pyrazol-1-yl)-phenol

To a solution of 4-[2-(4-Benzyloxy-phenyl-2H-pyrazol-3-yl]-pyridine (610 mg) in ethyl acetate (15 ml)/ethanol (15 ml) was added palladium hydroxide (20%, 343 mg). The reaction mixture was placed on a parr shaker under 45 psi of H₂ gas for 18 h. The reaction mixture was filtered through celite and concentrated. Purification via chromatotron (2 mm silica, 5% methanol/chloroform) provided the title compound (259 mg). MS: (M⁺H m/z=238.1).

### EXAMPLE 26

1-Methyl-2-[4-(5-pyridin-4-yl-pyrazol-1-yl)-phenoxymethyl]-1H-benzoimidazole

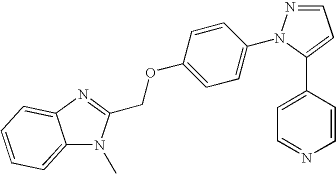

Following the procedure for the preparation of 1-Methyl-2-[4-(1-methyl-4-pyridin-4-yl-1H-pyrazol-3-yl)-phenoxymethyl-1H-benzoimidazole but substituting 4-(5-Pyridin-4-yl-pyrazol-1-yl-phenol provided the title compound. ¹H NMR (400 MHz, CDCl₃)δ 8.50 (d, J=6.2, 2H), 7.78 (d, J=7.5 Hz, 1H), 7.70 (s. 1H), 7.35 (s, 3H)₅ 7.20 (d, J=6.6 Hz, 2H), 7.08 (m, 4H), 6.60 (s, 1H) 5.40 (s, 2H), 3.89 (s, 3H); MS: (M⁺H m/z=382.1); PDE10 IC₅₀=3.05 nm.

Preparation 15

N-Methoxy-N-methyl-4-triisopropylsilanyloxmethyl-benzamide

Following the procedure for the preparation of 4-benzyloxy-N-methoxy-N-methyl-benzamide but substituting 4-Triisopropylsilanyloxmethyl-benzoic acid provided the title compound. MS: (M⁺H m/z=352.1).

Preparation 16

2-Pyridin-4-yl-1-(4-triisopropylsilanyloxmethyl-phenyl)-ethanone

Following the procedure for the preparation of 1-(4-Benzyloxy-phenyl)-2-pyridin-4-yl-ethanone but substituting N-Methoxy-N-methyl-4-triisopropylsilanyloxmethyl-benzamide provided the title compound. MS: (M⁺H m/z=384.1).

Preparation 17

4-[1-Methyl-3-(4-triisopropylsilanyloxmethyl-phenyl)-1H-pyrazol-4-yl]-pyridine

Following the procedure for the preparation of 4-[3-(4-Benzyloxy-phenyl)-1-methyl-1H-pyrazol-4-yl]-pyridine but substituting 2-Pyridin 4-yl-1-(4-triisopropylsilanyloxmethyl-phenyl)-ethanone provided the title compound. MS: (M⁺H m/z=422.2).

Preparation 18

[4-(1-Methyl-4-pyridin-4-yl-1H-pyrazol-3-yl)-phenyl]-methanol

To a solution of 4-[1-Methyl-3-(4triisopropylsilanyloxmethyl-phenyl)-1H-pyrazol-4-yl]-pyridine (1.75 g) in THF (16.2 mL) was added TBAF (1.0M THF, 5.2 mL) and the reaction mixture stirred at ambient temperature under inert atmosphere for 1 h. The reaction mixture was poured into saturated sodium bicarbonate, extracted 3× with chloroform, dried magnesium sulfate filtered and concentration. Purification via MPLC biotage chromatography eluting with 2% methanol/0.5% saturated ammonium hydroxide/50% ethyl acetate/hexanes provided the title compound (920 mg, 84%), MS: (M⁺H m/z=266.1).

Preparation 19

4-(1-Methyl-3-{4[triphenyl-15-phosphanyl)-methyl]-phenyl}-1H-pyrazol-4-yl)-pyridine

To a solution of [4-(1-Methyl-4-pyridin-4yl-1H-pyrazol-3-yl)-phenyl]-methanol (738 mg) in dioxane (14 mL) was added triphenyl phosphonium hydrogen bromide (1.94 g, 2 eq.) and the reaction mixture was heated at 100° C. for 2 h. The reaction mixture was cooled and filtered, dried in a vacuum oven to provide the title compound (1.75 g, 94%), MS: (M⁺H m/z=510.0).

Preparation 20

1-Methyl-2-{2-[4-(1-methyl-4-pyridin-4-yl-1H-pyrazol-3-yl)-phenyl]-vinyl}-1H-benzoimidazole

To a solution of 4-(1-Methyl-3-(4-[(triphenyl-15-phosphanyl)-methyl]-phenyl}-1H-pyrazol-4-yl)-pyridine bromide (300 mg) in dimethyl formamide (3 mL) was added cesium carbonate (290 mg, 3 eq.) and 1-Methyl-1H-benzoimidazole-2-carbaldehyde (52 mg) and the reaction mixture was heated at 40° C. for 3 h. The reaction mixture was poured into 1 N sodium hydroxide, extracted 3× chloroform, dried magnesium sulfate, filtered and concentrated. Purification via MPLC biotage chromatography eluting with 1-3% methanol/0.5% saturated ammonium hydroxide/80% ethyl acetate/hexanes provided the title compound. MS: (M⁺H m/z=392.0).

### EXAMPLE 27

1-Methyl-2{2-[4-(1methyl-4-pyridin-4-yl-1H-pyrazol-3-yl)-phenyl]-ethyl}-1H-benzoimidazole

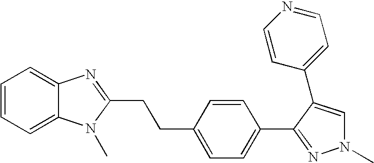

A solution of 1-Methyl-2-{2-[4-(1-methyl-4-pyridin-4-yl-1H-pyrazol-3-yl)-phenyl]-vinyl}-1H-benzoimidazole (100 mg) in ethanol (10 mL)/ethyl acetate (5 mL) in a parr bottle with palladium hydroxide (75 mg) was placed under 10 PSI of H₂ for 40 min. The reaction mixture was filtered and concentrated. Purification via MPLC biotage chromatography eluting with 1-3% methanol/0.5% saturated ammonium hydroxide/chloroform provided the title compound (88 mg). ¹H NMR (400 MHz, CDCl₃) δ 8.46 (m, 2 H;), 7.73 (m, 2H), 7.57 (s, 1H), 7.38 (d, J=7.9 Hz, 2H), 7.24 (m, 3H). 7.18 (m, 4H), 3.97 (s, 3H), 3.55 (s. 3H), 3.21 (m, 4H); MS: (M⁺H m/z=394.1). PDE10 IC₅₀=16.2 nm.

Preparation 21

4-(4-Pyridin-4-yl-4H-[1,2,4]triazol-3-yl)-phenol

To a solution of 4-Methoxy-pyridin-4-yl-benzamide (75 mg) in PCl₃ (3 ml) was added PCl₅ (68 mg) and the reaction mixture heated at reflux for 5 h. The reaction mixture was concentrated and dissolved in dimethyl formamide (2 ml) and Formic acid hydrazide (5 eq, 100 mg) was added and stirred for 2 h. The reaction mixture was concentrated and diluted with isopropanol (3 mL) and 0.25 ml of conc. HCl was added. The reaction mixture stirred for 18 h, quenched with 1 NaOH, extracted with dichloromethane, dried magnesium sulfate and concentrated. The crude product dissolved in methylene chloride (2 mL) and boron tribromide (0.63 mL 1.0M hexanes) was added at 0° C. The reaction mixture was warmed to ambient temperature and stirred for 18 h. The reaction mixture was quenched with 1 N NaOH and pH adjusted to 9, extracted with dichloromethane, dried magnesium sulfate, filtered and concentrated. Purification via Biotage MPLC chromatography eluting with 0-20% methanol/methylene chloride provided the title compound (32 mg, 55%). MS: (M⁺H m/z=239.2).

### EXAMPLE 28

1-Methyl-2[4-(4-pyridin-4-yl-4H-[1,2,4]triazol-3-yl)-phenoxymethyl]-1H-benzoimidazole

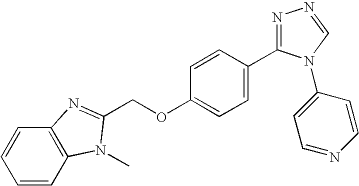

To a solution of 4-(4-Pyridin-4-yl-4H-[1,2,4]triazol-3-yl)-phenol (44 mg) in dimethyl formamide (1 ml) in a 7 ml Teflon capped vial was added cesium carbonate (185 mg) and 2-Chloromethyl-1-methyl-1H-benzoimidazole (38 mg) and the reaction mixture heated on a shaker plate at 60° C. for 18 h. The reaction mixture was poured into water and extracted with methylene chloride, dried magnesium sulfate, filtered and concentrated to provide the title compound (51 mg). ¹H NMR (400 MHz, CD₃OD) δ 8.88 (s, 1H), 8.64 (d, J=6,6 Hz, 2H), 7.64 (d, J=7.9 Hz, 1H), 7.54 (d, J=8.3 Hz, 1H), 7.40 (m, 4H), 7.36 (t, J=7.5 Hz, 1 H), 7.32 (t, J=7.5 Hz, 1H), 7.18 (d, J=8.7 Hz, 2H), 5.44 (s, 2H), 3.90 (S, 3H); MS: (M⁺H m/z 383,2); PDE10 IC₅₀=46.3 nm.

### EXAMPLE 29

2-Methyl-7-[4-(1-methyl-4-pyridin-4-yl-1H-pyrazol-3-yl)-phenoxymethyl]-thiazolo[3,2-a]pyrimidin-5-one

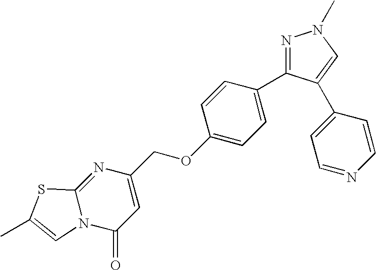

Following the procedure for the preparation of 2-[4-(1-Methyl-4-pyridin-4-yl-1H-pyrazol-3-yl)-phenoxymethyl]-benzothiazole but substituting 7-Chloromethyl-2-methyl-thiazolo[3,2-a]pyrimidin-5-one provided the title compound. ¹H NMR (400 MHz, CD₃OD) δ 8.34 (d, J=6.6 Hz, 2H), 7.98 (s 1H), 7.75 (m, 1H), 7.34 (d, J=8.7 Hz, 2H), 7.26 (d, J=6.2 Hz, 2H), 7.02 (d, J=9.1 Hz, 2H): 6.41 (s, 1H;), 5.00 (s, 2H), 3.94 (s, 3H), 2.45 (s, 3H); MS: (M⁺H m/z=430.1).

### EXAMPLE 30

7-[4-(1-Methyl-4-pyridin-4-yl-1H-pyrazol-3-yl-phenoxymethyl]-thiazolo-3,2-a]pyrimidin-5-one

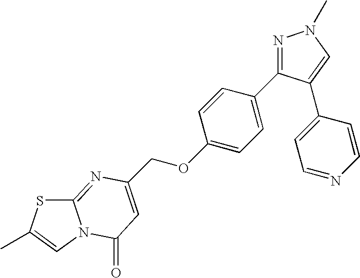

Following the procedure for the preparation of 2-[4-(1-Methyl-4-pyridin-4-yl-1H-pyrazol-3-yl)-phenoxymethyl]-benzothiazole but substituting 7-Chloromethyl-thiazolo[3,2-a]pyrimidin-5-one provided the title compound. ¹H NMR (400 MHz, CD₃OD) δ 8.40 (d, J=6.6 Hz, 2H), 8.07 (s, 1H), 8.03 (d, J=5.0 Hz, 1 H): 7.44 (d, J=5.0 Hz, 1 H), 7.37 (m, 4H), 7.06 (d, J=8.7 Hz, 2H), 6.45 (s, 1H): 5.06 (s, 2H): 3.95 (s, 3H); MS: (M⁺H m/z=416.1).

Preparation 22

4-Benzyloxy-2fluoro-benzoic acid benzyl ester

Following the procedure for the preparation of 4-(1-Methyl-1H-benzoimidazol-2-ylmethoxy)-benzoic acid methyl ester but substituting two equivalents of benzyl bromide and 2-Fluoro-4-hydroxy-benzoic acid provided the title compound. MS: (M⁺H m/z=337.2).

Preparation 23

4-Benzyloxy-2-fluoro-benzoic acid

Following the procedure for the preparation of 4-(1-Methyl-H-benzoimidazol-2-ylmethoxy)-benzoic acid but substituting 4-Benzyloxy-2-fluoro-benzoic acid benzyl ester provided the title compound. MS: (M⁺H m/z=247.1).

Preparation 24

4-Benzyloxy-2-fluoro-N-methoxy-N-methyl-benzamide

Following the procedure for the preparation of 4 N-Methoxy-N-methyl-4-(1-methyl-1H-benzoimidazol-2-yl-methoxy)-benzamide but substituting 4-Benzyloxy-2-fluoro-benzoic acid provided the title compound. MS: (M⁺H m/z=290.2).

Preparation 25

1-(4-Benzyloxy-2-fluoro-phenyl)-4-2-pyridin-4-yl-ethanone

Following the procedure for the preparation of 1-(4-Benzyloxy-phenyl)-2-pyridin-4-yl-ethanone but substituting 4-Benzyloxy-2-fluoro-N-methoxy-m-methyl-benzamide provided the title compound. MS: (M⁺H m/z=322.1).

Preparation 26

4-[3-(4-Benzyloxy-2-fluoro-phenyl)-1-methyl-1-H-pyrazol-4-yl]-pyridine

Following the procedure for the preparation of 4-[3-(4-Benzyloxy-phenyl)-1-methyl-1H-pyrazol-4-yl]-pyridine but substituting 1-(4-Benzyloxy-2-fluoro-phenyl)-2-pyridin-4-yl-ethanone provided the title compound. MS: (M⁺H m/z=360.1).

Preparation 27

3-Fluoro-4-(1-methyl-4-pyridin-4-yl-1H- pyrazol-3-yl)-phenol

Following the procedure for the preparation of 4-(1-Methyl-4-pyridin-4-yl-1H-pyrazol-3-yl)-phenol but substituting 4-[3-(4-Benzyloxy-2-fluoro-phenyl)-1-methyl-1H-pyrazol-4-yl]-pyridine provided the title compound. MS: (M⁺H m/z=270.1).

### EXAMPLE 31

2-[3-Fluoro-4-(1-methyl-4-pyridin-4-yl-1H-pyrazol-3yl)-phenoxymethyl]-1-methyl-1H-benzoimidazole

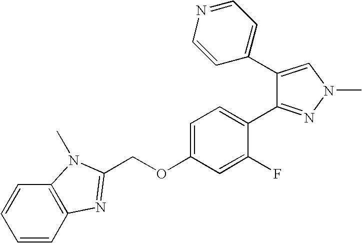

Following the procedure for the preparation of 1-Methyl-2-[4-(2-methyl-4-pyridin-4-yl-2H-pyrazol-3-yl)-phenoxymethyl]-1H-benzoimidazole but substituting 3-Fluoro-4-(1-methyl-4-pyridin-4-yl-1H-pyrazol-3-yl)-phenol provided the title compound, ¹H NMR (400 MHz, CDCl₃) δ 8,43 (d, J=6.2 Hz, 2H), 7.78 (d, J=7.5 Hz, 1H), 7.65 (s, 1H), 7.36 (m, 4H), 7.08 (d, J=6.2 Hz, 2H), 6.96 (dd, J=7.9, 2.1 Hz, 1H) 6.82 (dd, J=11.6, 2.9 Hz, 1H): 5.39 (s, 2H), 3.97 (s, 3H), 3.89 (s, 3H); MS: (M⁺H m/z=414.1).

### EXAMPLE 32

6-[4-(1-Methyl-4-pyridin-4-yl-1H-pyrazol-3-yl)-phenoxymethyl]-imidazo[2,1-b]thiazole

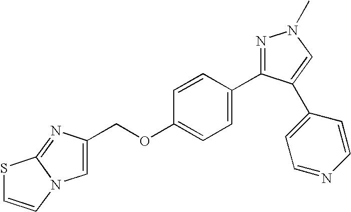

Following the procedure for the preparation of 2-[4-(1-Methyl-4-pyridin-4-yl-1H-pyrazol-3-yl)-phenoxymethyl]-benzothiazole but substituting 6-Chloromethyl-imidazo[2,1-b]thiazole provided the title compound. ¹H NMR (400 MHz, CD3OD) δ 8.43 (d, J=5.4 Hz, 2H), 7.98 (s, 1H) 7.75 (s, 1H), 7.71 (d, J=4.2 Hz, 1H), 7.32 (d, J=8.7 Hz, 2H), 7.25 (m, 2H), 7.12 (d, J=4.6 Hz, 2H), 7.04 (d, J=8.7 Hz, 2H), 5.46 (s, 2H), 3,94 (s, 3H); MS. (M⁺H m/z=388.3); PDE10 IC₅₀=12 nm.

Preparation 28

4-(1-(4-methoxyphenyl)-1H-imidazol-5-yl)pyridine

4-Methoxyaniline (2.46 g 20 mmol) and pyridine-4-carboxaldehyde (1.9 mL, 10 mmol) in toluene (110 mL) in a flask attached to a Dean-Stark trap and reflux condensor was heated at reflux. After 40 hours, the reaction was complete by infrared spectral analysis and mass spectral analysis. The toluene was removed via distillation through the Dean-Stark sidearm, the residue was dissolved in methanol (100 mL) and ca. ½ of the crude imine (ca. 10 mmol 50 mL of methanol solution) was diluted with methanol (20 mL) and 1 2-dimethoxyethane (20 mL). The solution was then treated with potassium carbonate (2.76 g 20 mmol) and tosylmethylisocyanide (TOSMIC, 2.93 g, 15 mmol) and was heated at reflux for 3 hours. After cooling to room temperature, the solvent was removed in vacuo, and the residue was dissolved in methylene chloride and was washed with brine. The brine layer was extracted with methylene chloride and the combined organic layers were dried (MgSO₄), were filtered, and were concentrated in vacuo. The residue was purified by silica gel chromatography with ethyl acetate-hexanes-methanol (80:20:0 to 76:19:5) to afford 1.4 g (56% yield) of the title compound; diagnostic ¹³C NMR signals (100 MHz, CDCl₃) δ 160.039, 150.161, 141.009, 137.240, 130.839, 129.179, 127.287, 121.597, 115.106, 55.801; MS (AP/Cl) 252.4 (M⁺H)⁺.

Preparation 29

4-(1-(4-benzyloxy)phenyl)-1-H-imidazol-5-yl)pyridine

The title compound was prepared using the method described for Preparation 28, substituting 4-benzyloxyaniline for 4-methoxyaniline, and afforded 4-(1-(4-(benzyloxy)phenyl)-1H-imidazol-5-yl)pyridine in 54% yield; diagnostic ¹³C NMR signals (100 MHz, CDCl₃) δ 159.195, 150.132, 141.001, 137.263, 136.403, 130.892, 130.735, 129.389, 128.932, 128.521, 127.751. 127.317, 121.627, 116.078, 70.637: MS (AP/Cl) 328.4 (M⁺H)⁺.

Preparation 30

4-(1-(4-methoxyphenyl)-2-methyl-1H-imidazol-5-yl)pyridine

A solution of diisopropyl amine (0.51 mL, 3.6 mmol) in tetrahydrofuran (12 mL) at −20° C., was treated with n-butyl lithium (2.5 M in hexanes, 1.45 mL. 3.6 mmol) and the solution was stirred for 10 minutes. A solution of Preparation 28 (4-(1-(4-methoxyphenyl)-1H-imidazol-5-yl)pyridine, 730 mg, 2.9 mmol) in tetrahydrofuran was added and the solution became dark orange. The solution was stirred for 30 minutes as the temperature was allowed to rise to 0° C. After cooling to −20° C., methyl iodide (0.54 mL, 8.7 mmol) in tetrahydrofuran (12 mL) was added and the solution was stirred for 30 min at −20° C. and for 2 hr at 23° C. The solvent was removed in vacuo, the residue was diluted with brine and was extracted with ethyl acetate. The organic layer was then dried (MgSO₄), was filtered, and was concentrated in vacuo. The residue was purified by silica gel chromatography using ethyl acetate-hexanes-methanol (63:32:5 to 72:18:10) to afford 555 mg (72% yield) of the title compound; diagnostic ¹³C NMR signals (100 MHZ, CDCl₃) δ 160.144, 150.034, 149.197, 137.749, 131.265, 129.463, 128.985, 128.828, 120.849, 115.233, 55.78. 14.203; MS (AP/Cl) 266.4 (M⁺H)⁺.

Preparation 31

4-(2-ethyl-1-(4-methoxyphenyl)-1H-imidazol-5-yl)pyridine

The title compound was prepared using the method described for Preparation 30 with ethyl iodide used in the place of methyl iodide and afforded 83% yield of 4-(2-ethyl-1-(4-methoxyphenyl)-1H-imidazol-5-yl)pyridine; diagnostic ¹³C NMR signals (100 MHz, CDCl₃) δ 160.144, 150.147, 149.990, 137.786, 129.239, 129.037, 128.992, 121.597, 120.909, 115,181, 55.771, 21.097, 12.348; MS (AP/Cl) 280.5 (M⁺H)⁺.

Preparation 32

4-(5-(pyridin-4-yl)-1-H-imidazol-1-yl)phenol

A solution of Preparation 29 (4-1-(4-(benzyloxy)phenyl)1H-imidazol-5-yl)pyridine, 2 g, 6.1 mmol) and anisole (13 mL, 122 mmol) in trifluoracetic acid (50 mL) was heated at 75° C. for 24 h. The solvent was removed in vacuo and the residue was purified via silica gel chromatography with chloroform-methanol-ammonium hydroxide (94:5:1) to afford 1.27 g (88%) of the title compound; diagnostic ¹³C NMR signals (100 MHz, CDCl₃) □158.402, 149.145, 141.061, 138.018, 120.600, 129.822, 127,482, 127.370, 121.933, 116.497; MS (AP/Cl) 238.3 (M⁺H)⁺.

Preparation 33

4-(2-methyl-5-(pyridin-4-yl)-1H-imidazol-1-yl)phenol

A solution of boron tribromide (1 M in methylene chloride, 2.1 mL, 2.1 mmol) was added dropwise to a solution of Preparation 30 (4-(1-(4-methoxyphenyl)-2-methyl-1H-imidazol-5-yl)pyridine, 220 mg, 0.83 mmol) in methylene chloride (5 mL) at 0° C. After stirring at 23° C. for 24 h. aqueous sodium hydroxide solution (1 N. 15 ml) was added and the mixture was stirred at 23° C. for 1 h. The pH was adjusted to 7 by the addition of aqueous hydrochloric acid (1 N), the mixture was extracted with methylene chloride/isopropanol (4:1, 3×30 mL), the combined organic layers were dried (MgSO₄), were filtered, and were concentrated in vacuo. The residue was purified by silica gel chromatography using chloroform-methanol (20:1 to 10:1) to afford 150 mg (722% yield ) of the title compound; diagnostic ¹³C NMR signals (100 MHz: CDCl₃) δ 159.337, 149.548, 149.302, 138.302, 131.131, 128.760, 128.170, 127.310, 121.163, 117.237, 13.881; MS (AP/Cl) 252.4 (M⁺H)⁺.

Preparation 34

4-(2-ethyl-5-(pyridin-4-yl)-1H-imidazol-1-yl)phenol

The title compound was prepared using Preparation 31 as the starting material and the method for Preparation 33. This yielded 4-(2-ethyl-5-(pyridin-4-yl)-1H-dimidazol-1-yl)phenol in 70% yield; diagnostic ¹³C NMR signals (100 MHz, CD₃OD/CDCl₃) δ 158.574, 149.182, 149.002, 138.511, 130.877, 128.895, 128.200, 127.340, 121.253, 116.692, 20.656, 12.020; MS (AP/Cl) 266.4 (M⁺H)⁺.

### EXAMPLE 33

2-((4-(5-(pyridin-4-yl)-1H-imidazol-1-yl)phenoxy)methyl)-1-methyl-1-H-benzo[d]imidazole

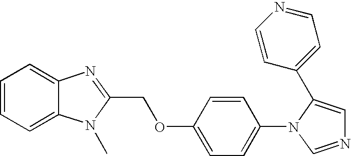

The title compound was prepared using the method described for the preparation of 2-[4-(1-Methyl-4-pyridin-4-yl-1H-pyrazol-3-yl)-phenoxymethyl]-benzothiazole with the substitution of 2-(chloromethyl)-1-methyl-1H-benzo[d]imidazole; 98% yield; diagnostic ¹³C NMR signals (100 MHz, CDCl₃) □158.290, 150.109, 148,913, 142.340, 140.942, 137.203, 136.388, 130.966, 130.682, 130.121, 127.407, 123.735, 122.801, 121.664, 120.430, 116.250, 109.692, 63.899, 30.549; MS (AP/Cl) 382.3 (M⁺H)⁺.

### EXAMPLE 34

2-((4-(5-(pyridin-4-yl)-1H-imidazol-1-yl)phenoxy)methyl)-1H-benzo]d]imidazole

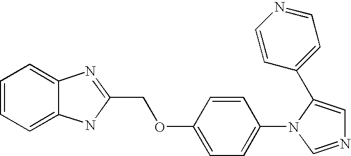

A solution of Preparation 32 (4-(5-(pyridin-4-yl)-1H-imidazol-1-yl)phenol, 0.5 g, 2.11 mmol) in N,N-dimethylformamide (DMF) (5 mL) was added dropwise to a suspension of sodium hydride (60% in mineral oil, 93 mg, 2.32 mmol) in DMF (10 mL) and was stirred at 23° C. for 10 min. A solution of 2-(chloromethyl)-1H-benzo[d]imidazole in OMF (10 mL) was added dropwise, then the reaction mixture was heated at 80° C. for 24 h. The solvent was removed in vacuo, the residue was diluted with water and was then extracted with methylene chloride. The organic layer was dried (MgSO₄), was filtered, and was concentrated in vacuo. Purification by silica gel chromatography using chloroform/methanol/ammonium hydroxide (98.5:1:0.5) gave 158 mg (20% yield) of the title compound; diagnostic ¹³C NMR signals (100 MHz, CDCl₃) δ 158.350, 150.004, 149.623. 140.919, 137.233, 130.854, 130.039, 127.385, 123,107, 121.716, 116.041, 65.066; MS (AP/Cl) 368.49 (M⁺H)⁺.

### EXAMPLE 35

2-((4-(2-methyl-5-(pyridin-4-yl)-1-H-imidazol-1-yl)phenoxy)methyl)-1-methyl-1-H-bezo[d]imidazole

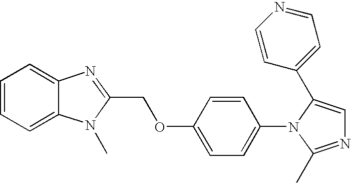

The title compound was prepared using the method described in Example 33 with the substitution of Preparation 33 for Preparation 32 and 2-(chloromethyl)-1-methyl-1H-benzo[d]imidazole; 93% yield; diagnostic ¹³C NMR signals (100 MHz, CDCl₃) δ 158.492, 150.027, 149.115, 148.935, 142.392, 137.644, 136.410, 131.250, 130.413, 129.030, 123.728, 122.786, 120.916, 120.453, 116.318, 109.707, 63.922, 30.564, 14.255; MS (AP/Cl) 396.4 (M⁺H)⁺.

### EXAMPLE 36

2-((4-(2-ethyl-5-(pyridin-4-yl)-1-imidazol-1-yl)phenoxy)methyl)-1-methyl-1H-benzo[d]imidazole

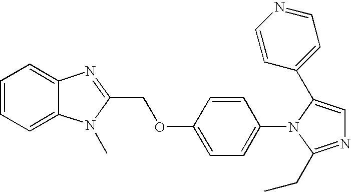

The title compound was prepared using the method described in Example 33 with the substitution of Preparation 34 for Preparation 32 and 2-(chloromethyl)-1-methyl-1H-benzo[d]imidazole; 46% yield; diagnostic ¹³C NMR signals (100 MHz, CDCl₃) δ 158.559, 153,833. 150.132, 149.900, 148.928, 137.719, 131.153, 130.129, 129.194, 128.947, 127.407, 123.758, 122.816, 121.672, 121.021, 120.453, 116.303, 109.715, 63.914, 30.579, 21.060, 12.318; MS (AP/Cl) 410.5 (M⁺H)⁺.

Preparation 35

N-(4-methoxyphenyl)isonicotinamide

A solution of p-anisidine (2.46 g, 20 mmol) and triethylamine (13.9 mL, 100 mmol) in ethyl acetate (200 mL) was treated with isonicotinic acid (2.46 g, 20 mmol) followed by 1-propanephosphonic acid cyclic anhydride (50% in ethyl acetate, 15.1 mL, 24 mmol). After stirring at 23° C. for 4 h, the reaction mixture was diluted with ethyl acetate, was washed with water and with brine, and the organic layer was dried (MgSO₄), was filtered, and was concentrated in vacuo. Purification by silica gel chromatography with chloroform-methanol (40:1) gave 4 g (88% yield) of the title compound; diagnostic ¹³C NMR signals (100 MHz, CD₃OD, CDCl₃) δ 164.825, 157.213, 149.758, 143.349, 130.989, 123.085, 122.068, 55,285; MS (AP/Cl) 229.3 (M⁺H)⁺.

Preparation 36

4-(1-(4-methoxyphenyl)-1H-imidazol-2-yl)pyridine

Preparation 35 (N-(4-methoxyphenyl)isonicotinamide,1 g, 4.39 mmol) was dissolved in phosphorous oxychloride (POCl₃) (5 mL) then phosphorous pentachloride (913 mg, 4.39 mmol) was added. The mixture was heated at 120° C. for 4 h. The POCl₃ was removed in vacuo, aminoacetaldehyde dimethyl acetal (9.5 mL, 87.8 mmol) and isopropanol (10 mL) were added, and the mixture was stirred at 23° C. for ca. 16 h. The reaction mixture was concentrated in vacuo and concentrated hydrochloric acid (36.5%, 25 mL) in isopropanol (15 mL) was added. The reaction mixture was heated at 90° C. for 24 h. After cooling to 23° C., aqueous sodium hydroxide (1N) and aqueous sodium bicarbonate were added to obtain pH=8. The mixture was extracted with methylene chloride, was dried (MgSO₄), and was filtered and concentrated in vacuo. The residue was purified by silica gel chromatography with ethyl acetate/hexanes/methanol (80:20:0 to 76:19:5) to afford 811 mg (74% yield) of the title compound; diagnostic ¹³C NMR signals (100 MHz, CDCl₃) δ 160.069, 149.952, 144.142, 137.853, 131.004, 129.882, 127.414, 124.977, 122.195, 115.114, 55.808; MS (AP/Cl) 252.4 (M⁺H)⁺.

Preparation 37

4-(2-(pyridin-4-yl)-1H-imidazol-1-yl)phenol

The title compound was prepared using the method outlined in Preparation 33 with the substitution of Preparation 36 for Preparation 30; 86% yield; diagnostic ¹³C NMR signals (100 MHz, CD₃OD/COCl₃) δ 158.372, 149.145, 143,641, 138.257, 129.232, 128.985, 127.347, 125.418, 122,666, 116.505; MS (AP/Cl) 238.4 (M+H)⁺.; PDE10 IC₅₀8.82 nm.

### EXAMPLE 37

2-((4-(2-(pyridin-4-yl)-1H-imidazol-1-yl)phenoxy)methyl)-1-methyl-1H-benzo[d]imidazole

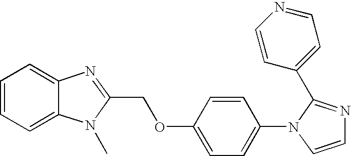

The title compound was prepared using the method outlined in Example 33 with the substitution of Preparation 37 for Preparation 32 and the substitution of 2-(chloromethyl)-1-methyl-1H-benzo[d]imidazole; 98% yield; diagnostic ¹³C NMR signals (100 MHz, CDCl₃) □ 158.312, 149.967, 148.913, 137.778, 131.961, 129.9797 127.579, 124.880, 123.758, 122.816, 122.247, 120.430, 116,265, 109.707, 63.899, 30.564; MS (AP/Cl) 382.4 (M+H)⁺.; PDE10 IC₅₀=28.8 nm.

The invention described and claimed herein is not to be limited in scope by the specific embodiments herein disclosed, since these embodiments are intended as illustrations of several aspects of the invention. Any equivalent embodiments are intended to be within the scope of this invention, indeed, various modifications of the invention in addition to those shown and described herein will become apparent to those skilled in the art from the foregoing description. Such modifications are also intended to fall within the scope of the appended claims.

1. A compound of formula I or a pharmaceutical acceptable salt thereof, 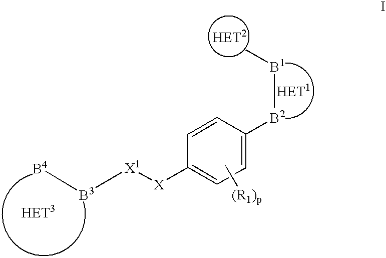
wherein HET¹ is selected from the group consisting of a monocyclic heteroaryl and a bicyclic heteroaryl, wherein said HET¹ may optionally be substituted with at least one R⁴;
HET² is a monocyclic heteroaryl, wherein said HET² may optionally be substituted with at least one R⁵;
HET³ is an 8 or 9 membered bicyclic heteroaryl, wherein said HET³ may optionally be substituted with at least one R⁶;
R¹ is selected from the group consisting of halogen, hydroxyl, cyano, C₁ to C₈ alkyl, C₂ to C₈ alkenyl, C₁ to C₈ alkynyl, C₁ to C⁸ alkoxy, C₁ to C₈ haloalkyl, C₃ to C₈ cycloalkyl, C₂ to C₇ heterocycloalkyl, C₁ to C₈ alkylthio, —NR³R³, —O—CF₃, —S(O)ₙ—R³, —C(O)—NR³R³, and C₁ to C₈ alkyl substituted with a heteroatom wherein the heteroatom is selected from the group consisting of nitrogen, oxygen and sulfur and wherein the heteroatom may be further substituted with one or more substituents selected from the group consisting of hydrogen, C₁ to C₈ alkyl, C₃ to C₅ cycloalkyl, C₂ to C₈ alkenyl, C₂ to C₈ alkynyl, and C₁ to C₈ haloalkyl;
each R² is independently selected from the group consisting of hydrogen, C₁ to C₈ alkyl, —C₃ to C₈ cycloalkyl-C₁ to C₈ alkyl, C₂ to C₈ alkenyl, C₂ to C₈ alkynyl, C₂ to C₈ alkenyl, C₁ to C haloalkyl and C₃ to C₈ cycloalkyl;
each R³ is independently selected from the group consisting of hydrogen, C₁ to C₈ alkyl C₂ to C₈ alkenyl, C₂ to C₈ alkynyl, C₁ to C₈ haloalkyl, C₃ to C₈ cycloalkyl;
each R⁴ is independently selected from the group consisting of halogen, hydroxyl, cyano, C₁ to C₈ alkyl, C₂ to C₈ alkenyl, C₂ to C₈ alkynyl, C₁ to C₈ alkoxy, C₃ to C₈ cycloalkyl, C₁ to C₈ alkylthio, C₁ to C₈ haloalkyl and C₁ to C₈ alkyl substituted with one or more substituents selected from the group consisting of —OR⁸, —NR⁸R⁸, and —SR⁸;
R⁶ is independently selected from the group consisting of halogen, hydroxyl, cyano,
—NR⁸R⁸, C₁ to C₈ alkyl, C₂ to C₈ alkenyl, C₂ to C₈ alkynyl, C₁ to C₈ alkoxy, C₃ to C₈ cycloalkyl, C₁ to C₈ alkylthio, and C₁ to C₈ haloalkyl;
B¹ and B² are adjacent atoms in Het¹ which are independently selected from the group consisting of carbon and nitrogen;
B³ and B⁴ are adjacent atoms in Het³ wherein B³ is carbon and B⁴ is nitrogen;
X and X¹ are each independently selected from the group consisting of oxygen, sulfur, —C(R₂)₂ and —NR₂, provided that at least one of X or X¹ is —C(R₂)₂;
wherein each R⁶ is independently selected from the group consisting of halogen, hydroxyl, cyano, C₁ to C₈ alkyl, C₂ to C₈ alkenyl, C₂ to C₈ alkynyl, C₁ to C₈ alkoxy, C₁ to C₈ cycloalkyl, C₁ to C₈ alkylthio, C₃ to C₈ haloalkyl, NR⁷R⁷, —O—CF₃, —S(O)ₘ—R⁷, and —C(O)NR⁷R⁷, C₁ to C₈ alkyl substituted with a heteroatom wherein the heteroatom is selected from the group consisting of nitrogen, oxygen and sulfur and wherein the heteroatom may be further substituted with a substituent selected from the group consisting of hydrogen, C₁ to C₈ alkyl, C₁ to C₈ cycloalkyl, C₂ to C₈ alkenyl, C₂ to C₈ alkynyl, and C₁ to C₈ haloalkyl;
or two R⁶'s together with the atoms which they are attached may optionally form a C₄ to C₁₀ cycloalkyl, C₄ to C₁₀ cycloalkenyl, (4-10 membered) heterocycloalkyl or (4-10 membered) heterocycloalkenyl ring;
wherein each R⁷ is independently selected from the group consisting of hydrogen and C₁-C₈ alkyl;
wherein each R⁸ is independently selected from the group consisting of hydrogen, C₁ to C₈ alkyl, C₂ to C₈ alkenyl and C₂ to C₈ alkynyl;
n=0, 1 or 2; m=0, 1 or 2; and p=0, 1, 2, 3 or 4.

2. The compound of claim 1, wherein said HET³ is selected from the group consisting of: 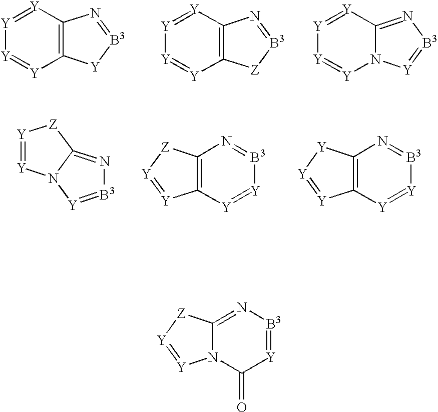
wherein each Y is independently selected from the group consisting of —CH, —CR⁶ or nitrogen:
and Z is oxygen or sulfur.

3. The compound of claim 2, wherein all Y's are independently —CH or —CR⁶.

4. The compound of claim 1, wherein said HET³ is selected from the group consisting of:

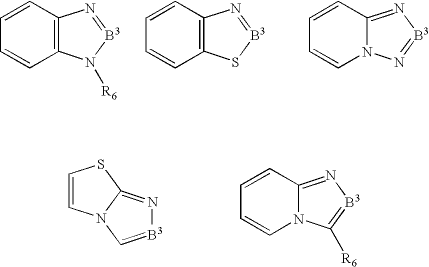

5. The compound of claim 1, wherein HET¹ is a 5 membered heteroaryl.

6. The compound of claim 1, wherein HET¹ is selected from the group consisting of pyrazole, isoxazolyl, triazolyl, oxazolyl, thiazolyl and imidazolyl.

7. The compound of claim 1, wherein HET² is selected from the group consisting of 4-pyridyl, 4-pyridazinyl and isoxazolyl.

8. The compound of claim 1, wherein HET² is 4-pyridyl.

9. The compound of claim 1, wherein HET¹ is selected from the group consisting of: 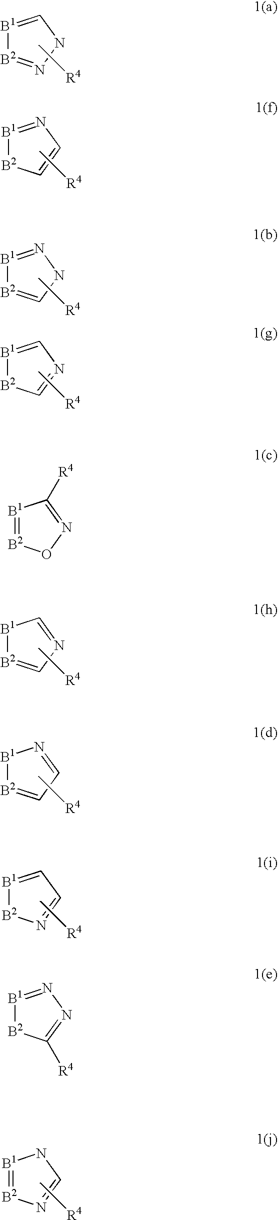
wherein in 1(a), B¹ and B² are carbon;
wherein in 1(b), B¹ and B² are carbon:
wherein in 1(c), B¹ and B² are carbon;
wherein in 1(d), B¹ is nitrogen and B² is carbon;
wherein in 1(e), B¹ is carbon and B² is nitrogen;
wherein in 1(f), B¹ is carbon and B² is nitrogen;
wherein in 1(g), B¹ s carbon and B² is nitrogen;
wherein in 1(h), B¹ is nitrogen and B² is carbon;
wherein in 1(i), B¹ is nitrogen and B² is carbon; and
wherein in 1(j), B¹ is carbon and B² is carbon;

10. The compound of claim 9, wherein HET¹ is selected from the group 1a.

11. The compound of claim 10, wherein HET² is 4-pyridyl

12. The compound of claim 1, wherein X¹ is carbon and X is oxygen.

13. A compound selected from the group consisting of:
1-Methyl-2-[4-(4-pyridin-4-yl-1H-pyrazol-3-yl)-phenoxymethyl]-1H-benzoimidazole;
2-[4-(1-Ethyl-4-pyridin-4-yl-1H-pyrazol-3-yl)-phenoxymethyl]-1-methyl-1-H benzoimidazole;
1-{3-[4-(1-Methyl-1H-benzoimidazol-2-ylmethoxy)-phenyl]-4-pyridin-4-yl-pyrazol-1-yl}-propan-2-ol;
1-Methyl-2-[4-(4-pyridin-4-yl-isoxazol-5-yl)phenoxymethyl]-1H-benzoimidazole;
1-Methyl-2-[4-( 1-methyl-4-pyridin-4-yl-1H-pyrazol-3-yl)-phenoxymethyl]-1-H-benzoimidazole;
1-Methyl-2-[4-(2-methyl-4-pyridin-4-yl-2H-pyrazol-3-yl)-phenoxymethyl]-1-H-benzoimidazole;
1-Fluoromethyl-2-[4-(1-methyl-4-pyridin-4-yl-1-H-pyrazol-3-yl)-phenoxymethyl]-1H-benzoimidazole;
1-Isopropyl-2[4-(1-methyl-4-pyridin-4-yl-1H-pyrazol-3-yl)-phenoxymethyl]-1H-benzoimidazole;
1-Cyclopropyl-2-[4-(1-methyl-4-pyridin-4-yl-1H-pyrazol-3-yl)-phenoxymethyl]-1H-benzoimidazole;.
1-(2-Methoxy-ethyl)-2-[4-(1-methyl-4-pyridin-4-yl-1H-pyrazol-3-yl)-phenoxymethyl]-1H-benzoimidazole;
2-[4-(1-Methyl-4-pyridin-4-yl-1H-pyrazol-3-yl)-phenoxymethyl]-imidazo[1,2-a]pyridine;
2-[4-(2-Methyl-4-pyridin-4-yl-2H-pyrazol-3-yl)-phenoxymethyl]-imidazo[1,2-a]pyridine;
2-[4-(1-Methyl-4-pyridin-4-yl-1H-pyrazol-3-yl)-phenoxymethyl]-[1,2,4]triazolo[1,5-a]-pyridine;
2-[4-4-Pyridin-4-yl-1-(2,2,2-trifluoro-ethyl)-1H-pyrazol-3-yl]-phenoxymethyl}-[1,2,4]triazolo[1,5-a]pyridine;
2-{4-[4-Pyridin-4-yl-1-(2,2,2-trifluoro-ethyl)-1H-pyrazol-3-yl]-phenoxymethyl}-imidazo[1,2-a]pyridine;
1-Methyl-2{-[4-pyridin-4-yl-1-(2,2,2-trifluoro-ethyl)-1H-pyrazol-3]-yl-phenoxymethyl}-1H-benzoimidazole;
1-Fluoromethyl-2-{4-[4-pyridin-4-yl-1-(2,2,2-trifluoro-ethyl)-1-H-pyrazol-3-yl]-phenoxymethyl}-1H-benzoimidazole;
1-Methyl-2-[4-(1-methyl-4-pyridin-4-yl-1H-pyrazol-3-yl)-phenoxymethyl]-1H-imidazo[4,5-b]pyridine;
1-Methyl-2-[4-(1-methyl-4-pyridin-4-yl-1H-pyrazol-3-yl)-phenoxymethyl]-1H-imidazo[4,5-c]pyridine:
5,6-Difluoro-1-methyl-2-[4-(1-methyl-4-pyridin-4-yl-1H-pyrazol-3-yl)-phenoxymethyl]-1H-benzoimidazole;
2-[4-(1-Methyl-4-pyridin-4-yl-1H-pyrazol-3-yl)-phenoxymethyl]-benzothiazole;
2-{4-[4-Pyridin-4-yl-1-(2,2,2-trifluoro-ethyl)-1H-pyrazol-3-yl]-phenoxymethyl}-benzothiazole;
2-[4-(1-Methyl-4-pyridin-4-yl-1H-pyrazol-3-yl)-phenoxymethyl]-5,6-dihydro-4H-imidazo[4,5,1ij]quinoline;
3-Methyl-2-[4-(1-methyl-4-pyridin-4-yl-1H-pyrazol-3-yl)-phenoxymethyl]-imidazo[1,2-a]pyridine;
2-[4-(1-Methyl-4-pyridin-4-yl-1H-pyrazol-3-yl)-phenoxymethyl]-1-(2,2,2-trifluoro-ethyl)-1H-benzoimidazole;
1-Methyl-2-[4-(5-pyridin-4-yl-pyrazol-1-yl)-phenoxymethyl]-1H-benzoimidazole;
1-Methyl-2-{2-[4-(1-methyl-4-pyridin-4-yl-1H-pyrazol-3-yl)-phenyl]-ethyl}-1H-benzoimidazole;
1-Methyl-2-[4-(4-pyridin-4-yl-4H-[1,2,4]triazol-3-yl)-phenoxymethyl]-1H-benzoimidazole;
2-Methyl-7-[4-(1-methyl-4-pyridin-4-yl-1H-pyrazol-3-yl)-phenoxymethyl]-thiazolo[3,2-a]pyrimidin-5-one;
7-[4-(1-Methyl-4-pyridin-4-yl-1H-pyrazol-3-yl)-phenoxymethyl]-thiazolo[3,2-a]pyrimidin-5-one;
2-[3-Fluoro-4-(1-methyl-4-pyridin-4-yl-1H-pyrazol-3-yl)-phenoxymethyl]-1-methyl-1-H-benzoimidazole:
6-[4-(1-Methyl-4-pyridin-4-yl-1H-pyrazol-3-yl)-phenoxymethyl]-imidazo[2,1-b]thiazole;
2-((4-(5-(pyridin-4-yl)-1H-imidazol-1-yl)phenoxy)methyl)-1-methyl-1H-benzo[d]imidazole,
2-((4-(5-(pyridin-4-yl)-1H-imidazol-1-yl)phenoxy)methyl)-1H-benzo[d]imidazole;
2-((4-(2-methyl-5-(pyridin-4yl)-1-H-imidazol-1-yl)phenoxy)methyl-1-methyl-1H-benzo[d]imidazole;
2-((4-(2-ethyl-5-(pyridin-4-yl)-1H-imidazol-1-yl)phenoxy)methyl)-1-methyl-1H-benzo[d]imidazole;
2-((4-(2-(pyridin-4-yl)-1H-imidazol-1-yl)phenoxy)methyl)-1-methyl-1H-benzo[d]imidazole;
and pharmaceutical acceptable salts thereof.

14. A pharmaceutical composition for treating psychotic disorders, delusional disorders and drug induced psychosis; anxiety disorders, movement disorders, mood disorders, neurodegenerative disorders and drug addiction, comprising an amount of a compound of formula I according to claim 1 effective in treating said disorder or condition.

15. Use of the compound of formula I according to claim 1 in the manufacture of a medicament for treating a disorder selected from psychotic disorders, delusional disorders and drug induced psychosis; anxiety disorders, movement disorders, mood disorders, and neurodegenerative disorders.

16. The use of claim 15, wherein said disorder are selected from the group consisting of: dementia, Alzheimer's disease, multi-infarct dementia, alcoholic dementia or other drug-related dementia, dementia associated with intracranial tumors or cerebral trauma, dementia associated with Huntington's disease or Parkinson's disease, or AIDS-related dementia; delirium; amnestic disorder; post-traumatic stress disorder; mental retardation: a learning disorder, for example reading disorder, mathematics disorder, or a disorder of written expression; attention-deficit/hyperactivity disorder, age-related cognitive decline, major depressive episode of the mild, moderate or severe type; a manic or mixed mood episode: a hypomanic mood episode; a depressive episode with atypical features; a depressive episode with melancholic features, a depressive episode with catatonic features, a mood episode with postpartum onset; post-stroke depression; major depressive disorder; dysthymic disorder, minor depressive disorder; premenstrual dysphoric disorder; post-psychotic depressive disorder of schizophrenia; a major depressive disorder superimposed on a psychotic disorder comprising a delusional disorder or schizophrenia; a bipolar disorder comprising bipolar I disorder, bipolar II disorder, cyclothymic disorder, Parkinson's disease, Huntington's disease; dementia, Alzheimer's disease, multi-infarct dementia, AIDS-related dementia, Fronto temperal Dementia; neurodegeneration associated with cerebral trauma; neurodegeneration associated with stroke; neurodegeneration associated with cerebral infarct, hypoglycemia-induced neurodegeneration; neurodegeneration associated with epileptic seizure; neurodegeneration associated with neurotoxin poisoning; multi-system atrophy, paranoid, disorganized, catatonic, undifferentiated or residual type, schizophreniform disorder; schizoaffective disorder of the delusional type or the depressive type; delusional disorder; substance-induced psychotic disorder, psychosis induced by alcohol, amphetamine, cannabis, cocaine, hallucinogens, inhalants, opioids, or phencyclidine; personality disorder of the paranoid type; and personality disorder of the schizoid type.
